SEQUENCE LISTING (11 sequences)

SEQ ID NO: 1 - protein, 459 aa, Homo sapiens [sequence omitted]

SEQ ID NO: 2 - protein, 117 aa, Homo sapiens
EVQLVESGGGLVKPGGSLRLSCAASGFTFDDSVMHWVRQAPGKGLEWVSLVGWDGFFTYYADSVKGRFTISRDNAKNSLYLQMNSLRAEDTAVYYCARQGDYMGNNWGQGTLVTVSS

SEQ ID NO: 3 - protein, 110 aa, Homo sapiens
NFMLTQPHSVSESPGKTVTISCTRSSGSIDSSYVQWYQQRPGSSPTTVIYEDDQRPSGVPDRFSGSIDSSSNSASLTISGLKTEDEADYYCQSYDFHHLVFGGGTKLTVL

SEQ ID NO: 4 - protein, 10 aa, Homo sapiens
GFTFDDSVMH

SEQ ID NO: 5 - protein, 17 aa, Homo sapiens
LVGWDGFFTYYADSVKG

SEQ ID NO: 6 - protein, 8 aa, Homo sapiens
QGDYMGNN

SEQ ID NO: 7 - protein, 13 aa, Homo sapiens
TRSSGSIDSSYVQ

SEQ ID NO: 8 - protein, 7 aa, Homo sapiens
EDDQRPS

SEQ ID NO: 9 - protein, 9 aa, Homo sapiens
QSYDFHHLV

SEQ ID NO: 10 - protein, 432 aa, Homo sapiens
EVQLVESGGGLVKPGGSLRLSCAASGFTFDDSVMHWVRQAPGKGLEWVSLVGWDGFFTYYADSVKGRFTISRDNAKNSLYLQMNSLRAEDTAVYYCARQGDYMGNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 215 aa, Homo sapiens
NFMLTQPHSVSESPGKTVTISCTRSSGSIDSSYVQWYQQRPGSSPTTVIYEDDQRPSGVPDRFSGSIDSSSNSASLTISGLKTEDEADYYCQSYDFHHLVFGGGTKLTVLQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2017/0247460 A1
(43) Pub. Date: Aug. 31, 2017

(54) ANTI-IL-7R ANTIBODY COMPOSITIONS

(71) Applicant: PFIZER INC., New York, NY (US)

(72) Inventors: Monika Hildegard Pauline GEIGER, Somerville, MA (US); Davin Thomas BOARDMAN, Haverhill, MA (US); Robert Henry WALTERS, Medford, MA (US)

(21) Appl. No.: 15/519,803

(22) PCT Filed: Oct. 6, 2015
(86) PCT No.: PCT/IB2015/057636

§ 371 Date: Apr. 17, 2017

Related U.S. Application Data
(60) Provisional application No. 62/065,612, filed on Oct. 18, 2014.

(51) Int. Cl. C07K 16/28; A61K 47/18; A61K 47/26; A61K 39/395

(52) U.S. Cl. CPC C07K 16/2866; A61K 39/39591; A61K 47/183; A61K 47/26; C07K 2317/565; C07K 2317/56

(57) ABSTRACT

The present invention relates generally to the field of pharmaceutical formulations of antibodies. Specifically, the present invention relates to a high concentration antibody formulation and its pharmaceutical preparation and use. This invention is exemplified by a formulation of an anti-IL-7R antibody.

This application claims the benefit of U.S. Provisional Application No. 62/065,612 filed on Oct. 18, 2014, the contents of which is hereby incorporated by reference in its entirety.

### FIELD

The present invention relates to the field of pharmaceutical formulations of antibodies. Specifically, the present invention relates to an anti-IL7R antibody formulation and its pharmaceutical preparation and use.

### BACKGROUND

Antibody therapeutics are typically administered on a regular basis and generally involve several mg/kg dosing by injection. Parental delivery is a common route of administration for therapeutic antibody. Relatively high concentration antibody formulations are desirable for parental administration in order to minimize the volume of each dose.

Development of highly concentrated protein formulations can be a challenge due to issues relating to the physical and chemical stability of the protein, manufacture, storage, and delivery of the protein formulation. Increased viscosity of antibody formulations can cause problems from drug manufacture through drug delivery to the patient. Various attempts have been made to study the effect of viscosity-reducing agents on highly concentrated aqueous protein-containing formulations.

It has been shown that the anti-IL-7R antibody is useful in the treatment of type 2 diabetes, graft-versus-host disease (GVHD), and autoimmune disorders, including type 1 diabetes, multiple sclerosis, rheumatoid arthritis, and lupus (see for example WO2011/104687). There is a need for a stable, high concentration antibody preparation of an anti-IL-7R antibody having suitable viscosity to meet the medical need of patients suffering from conditions mediated by IL-7R.

### SUMMARY

Compositions comprising an IL-7R antibody and excipients capable of reducing the viscosity of a formulation comprising the antibody are provided. It is demonstrated that certain excipients are effective to reduce viscosity. Advantageously, the compositions provided herein demonstrate viscosity behavior suitable to achieve concentrations of greater than 100 mg/mL for a drug product to be used for therapeutic treatment.

Provided herein are anti-IL-7R antibody compositions which support high concentrations of bioactive antibody in solution and are suitable for parenteral administration, including intravenous, intramuscular, intraperitoneal, intradermal or subcutaneous injection. In some embodiments, the compositions can comprise an anti-IL-7R antibody, arginine HCl or NaCl, a tonicity agent, a buffer, a chelating agent, and a polysorbate. In some embodiments the pH of the composition can be between about 5.8 to 7.5.

In some embodiments, the composition can comprise or consist essentially of between about 100 mg/ml to about 200 mg/ml anti-IL-7R antibody, arginine HCl or NaCl, a tonicity agent, a buffer, a chelating agent, and a polysorbate, and has a pH of about 6.5 to about 7.5.

In some embodiments, the tonicity agent can be sucrose. In some embodiments the concentration of sucrose can be about 1 mg/ml to about 100 mg/ml. In some embodiments, the concentration of sucrose is about 50 mg/ml.

In some embodiments, the concentration of polysorbate can be from about 0.01 to about 0.3 mg/ml. In some embodiments, the concentration of polysorbate is about 0.2 mg/ml. In some embodiments, the the polysorbate is polysorbate 80.

In some embodiments, the buffer can be histidine buffer. In some embodiments, the concentration of histidine buffer can be from about 1.0 to about 30 mM. In some embodiments, the concentration of histidine buffer is about 20 mM histidine.

In some embodiments, the chelating agent can be disodium EDTA. In some embodiments, the concentration of disodium EDTA can be from about 0.01 to about 0.3 mg/mL. In some embodiments, the concentration of disodium EDTA can be from about 0.01 mg/mL, about 0.05 mg/mL, about 0.1 mg/mL, about 0.15 mg/mL, about 0.2 mg/mL, about 0.25 mg/mL, or about 0.3 mg/mL. In some embodiments, the concentration of EDTA is about 0.05 mg/mL.

In some embodiments, the antibody concentration can between about 100 mg/ml to about 150 mg/ml. In some embodiments, the antibody concentration can be about 130 mg/ml, about 135 mg/ml and about 140 mg/ml. In some embodiments, the antibody concentration is about 120 mg/ml.

In some embodiments, the arginine HCl concentration is about 100 mM.

In some embodiments, the composition comprises about 100 mg/ml to about 150 mg/ml of an antibody, about 50 to about 150 mM arginine HCl or NaCl, about 15 mM to about 30 mM histidine buffer, about 1 mg/ml to about 100 mg/ml sucrose, about 0.01 to about 0.25 mg/ml PS80, and about 0.01 to about 0.1 mg/ml. disodium EDTA, and the composition is of a pH from 6.5 to 7.5.

In some embodiments, the composition comprises about 10 mg/ml, about 105 mg/ml, about 110 mg/ml, about 115 mg/ml, about 120 mg/ml, about 125 mg/ml, about 130 mg/ml, about 135 mg/ml or about 140 mg/ml of an antibody, about 20 mM histidine buffer, about 100 mM arginine HCl or NaCl, about 50 mg/ml sucrose, about 0.2 mg/ml PS80, about 0.05 mg/ml disodium EDTA, and the composition is of a pH 7.0+/−0.5.

In some embodiments, the composition comprises or consists essentially of about 10 mg/ml, about 105 mg/ml, about 110 mg/ml, about 115 mg/ml, about 120 mg/ml, about 125 mg/ml, about 130 mg/ml, about 135 mg/ml or about 140 mg/ml of an antibody, about 20 mM histidine buffer, about 100 mM arginine HCl, about 50 mg/ml sucrose, about 0.2 mg/ml PS80, about 0.05 mg/ml disodium EDTA, and the composition is of a pH 7.0+/−0.5.

In some embodiments, the composition comprises or consists essentially of about 120 mg/ml of an antibody, about 20 mM histidine buffer, about 100 mM arginine HCl, about 50 mg/ml sucrose, about 0.2 mg/ml PS80, about 0.05 mg/ml disodium EDTA, and the composition is of a pH 7.0+/−0.5.

In some embodiments, the composition comprises or consists essentially of about 130 mg/ml of an antibody, about 20 mM histidine buffer, about 100 mM arginine HCl, about 50 mg/ml sucrose, about 0.2 mg/ml PS80, about 0.05 mg/ml disodium EDTA, and the composition is of a pH 7.0+/−0.5.

In some embodiments, the antibody can be a human or humanized monoclonal antibody. In some embodiments, the antibody can be an IgG1 or IgG2 antibody. In some embodiments, the antibody can bind to human IL-7Rα with a Kd of about 0.2 nM to about 2 nM. In some embodiments, the antibody can comprise a heavy chain CDR1, CDR2, CDR3, and a light chain CDR1, CDR2, and CDR3 comprising the amino acid sequence shown in SEQ ID NO: 4, 5, 6, 7, 8, and 9, respectively. In some embodiments, the antibody can comprise a variable heavy chain sequence comprising the amino acid sequence shown in SEQ ID NO: 10 and a variable light chain sequence comprising the amino acid sequence shown in SEQ ID NO: 11.

In some embodiments, the composition may not be lyophilized. In other embodiments, the composition may be lyophilized.

In some embodiments, the composition may have a viscosity of less than about 50 cP, less than about 40 cP, less than about 30 cP, or less than about 20 cP at 25° C. In some embodiments, the composition may have a viscosity of about 5 to about 50 cP at 25° C. In some embodiments, the composition may have a viscosity of about 5 to about 40 cP at 25° C. In some embodiments, the composition may have a viscosity of about 5 to about 30 cP at 25° C. In some embodiments, the composition may have a viscosity of about 5 to about 20 cP at 25° C.

Also provided herein is manufacture of a medicament for treatment of an autoimmune disorder in a mammal.

Also provided herein are uses of the composition for the manufacture of a medicament for treatment of an autoimmune disorder in a mammal. In some embodiments, the administration pattern of the medicament comprises administration of a dose of the medicament once every eight weeks. In some embodiments, the autoimmune disorder can be type 1 diabetes, multiple sclerosis, graft versus host disease, or lupus.

Also provided herein are uses of the composition for the preparation of a medicament for the treatment of of an autoimmune disorder in a mammal. In some embodiments, the autoimmune disorder can be type 1 diabetes, multiple sclerosis, graft versus host disease, or lupus.

Also provided herein are uses of the composition for the treatment of an autoimmune disorder in a mammal. In some embodiments, the autoimmune disorder can be type 1 diabetes, multiple sclerosis, graft versus host disease, or lupus.

In some embodiments, the volume of the dose can be less than or equal to about 2.5 ml, about 2.0 ml, about 1.5 ml, or about 1.0 ml. In some embodiments, administration of the dose can be intravenous. In some embodiments, administration of the dose can be subcutaneous.

In some embodiments, the mammal can be a human.

### BRIEF DESCRIPTION OF THE DRAWINGS

FIG. 1A depicts a graph comparing the viscosity of anti-IL-7R antibody formulation 1 at different pH values.

FIG. 1B depicts a graph comparing the viscosity of anti-IL-7R antibody formulation at different pH values

FIG. 2 depicts a graph comparing the viscosity of anti-IL7R antibody formulation with and without arginine HCl.

FIG. 3 depicts a graph comparing the viscosity of anti-IL-7R antibody formulation at different pH values.

FIG. 4 depicts a graph comparing the viscosity of anti-IL-7R antibody formulation at different pH values with 150 mM excipient addition.

FIG. 5 depicts a graph comparing the viscosity of anti-IL-7R antibody formulation at pH 5.9 and pH 7 with addition of 150 mM NaCl or 150 mM arginine HCl.

FIG. 6 depicts a graph comparing the viscosity of anti-IL-7R antibody formulation in 20 mM histidine buffer pH 7.0 with different concentrations of NaCl.

FIG. 7 depicts a graph comparing the viscosity of anti-IL-7R antibody formulation in 20 mM histidine buffer pH 7.0 with different concentrations of arginine HCl.

FIG. 8 depicts a graph comparing the viscosity of anti-IL-7R antibody formulations.

FIG. 9A depicts a graph comparing aggregation of anti-IL-7R antibody at 40° C.

FIG. 9B depicts a graph comparing aggregation of anti-IL-7R antibody at 2-8° C.

FIG. 10A depicts a graph comparing charge isoforms of anti-IL-7R antibody at 40° C.

FIG. 10B depicts a graph comparing charge isoforms of anti-IL-7R antibody at 2-8° C.

FIG. 11A depicts a graph comparing fragmentation of anti-IL-7R antibody at 40° C.

FIG. 11B depicts a graph comparing fragmentation of anti-IL-7R antibody at 2-8° C.

FIG. 12 depicts a graph comparing the turbidity (clarity) of anti-IL-7R antibody formulations.

### DETAILED DESCRIPTION

Disclosed herein are compositions having reduced viscosity. Advantageously, the compositions stably support high concentrations of bioactive antibody in solution and are suitable for parenteral administration, including intravenous, intramuscular, intraperitoneal, intradermal or subcutaneous injection.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

The following terms, unless otherwise indicated, shall be understood to have the following meanings: the term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

As used herein, the terms “formulation” or “composition” as they relate to an antibody are meant to describe the antibody in combination with a pharmaceutically acceptable excipient comprising at least one tonicity agent, at least one buffer, at least one chelating agent, at least one surfactant, wherein the pH is as defined.

The terms “pharmaceutical composition” or “pharmaceutical formulation” refer to preparations which are in such form as to permit the biological activity of the active ingredients to be effective.

“Pharmaceutically acceptable excipients” (vehicles, additives) are those, which can safely be administered to a subject to provide an effective dose of the active ingredient employed. The term “excipient” or “carrier” as used herein refers to an inert substance, which is commonly used as a diluent, vehicle, preservative, binder or stabilizing agent for drugs. As used herein, the term “diluent” refers to a pharmaceutically acceptable (safe and non-toxic for administration to a human) solvent and is useful for the preparation of the liquid formulations herein. Exemplary diluents include, but are not limited to, sterile water and bacteriostatic water for injection (BWFI).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)2, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “ABM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

As used herein, the term “human antibody” is intended to include antibodies having variable and constant regions derived from human germline immunoglobulin sequences. This definition of a human antibody includes antibodies comprising at least one human heavy chain polypeptide or at least one human light chain polypeptide. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; and 6,180,370.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349: 293-299 (1991), Lobuglio et al. Proc. Nat. Acad. Sci. USA 86: 4220-4224 (1989), Shaw et al. J Immunol. 138: 4534-4538 (1987), and Brown et al. Cancer Res. 47: 3577-3583 (1987). Other references describe rodent CDRs grafted into a human supporting framework region (FR) prior to fusion with an appropriate human antibody constant domain. See, for example, Riechmann et al. Nature 332: 323-327 (1988), Verhoeyen et al. Science 239: 1534-1536 (1988), and Jones et al. Nature 321: 522-525 (1986). Another reference describes rodent CDRs supported by recombinantly veneered rodent framework regions. See, for example, European Patent Publication No. 0519596. These “humanized” molecules are designed to minimize unwanted immunological response toward rodent anti-human antibody molecules which limits the duration and effectiveness of therapeutic applications of those moieties in human recipients. For example, the antibody constant region can be engineered such that it is immunologically inert (e. g., does not trigger complement lysis). See, e. g. PCT Publication No. WO99/58572; UK Patent Application No. 9809951.8. Other methods of humanizing antibodies that may also be utilized are disclosed by Daugherty et al., Nucl. Acids Res. 19: 2471-2476 (1991) and in U.S. Pat. Nos. 6,180,377; 6,054,297; 5,997,867; 5,866,692; 6,210,671; and 6,350,861; and in PCT Publication No. WO 01/27160.

As used herein, the term “recombinant antibody” is intended to include all antibodies that are prepared, expressed, created or isolated by recombinant means, for example antibodies expressed using a recombinant expression vector transfected into a host cell, antibodies isolated from a recombinant, combinatorial human antibody library, antibodies isolated from an animal (e.g., a mouse) that is transgenic for human immunoglobulin genes or antibodies prepared, such recombinant human antibodies can be subjected to in vitro mutagenesis.

The term “epitope” refers to that portion of a molecule capable of being recognized by and bound by an antibody at one or more of the antibody's antigen-binding regions. Epitopes often consist of a surface grouping of molecules such as amino acids or sugar side chains and have specific three-dimensional structural characteristics as well as specific charge characteristics. In some embodiments, the epitope can be a protein epitope. Protein epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds. The term “antigenic epitope” as used herein, is defined as a portion of an antigen to which an antibody can specifically bind as determined by any method well known in the art, for example, by conventional immunoassays. Once a desired epitope on an antigen is determined, it is possible to generate antibodies to that epitope, e.g., using the techniques described in the present specification. Alternatively, during the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another for binding to IL-7R, e.g., the antibodies compete for binding to the antigen.

As used herein, the terms “isolated antibody” or “purified antibody” refers to an antibody that by virtue of its origin or source of derivation has one to four of the following: (1) is not associated with naturally associated components that accompany it in its native state, (2) is free of other proteins from the same species, (3) is expressed by a cell from a different species, or (4) does not occur in nature.

An antibody is “substantially pure,” “substantially homogeneous,” or “substantially purified” when at least about 60 to 75% of a sample exhibits a single species of antibody. A substantially pure antibody can typically comprise about 50%, 60%, 70%, 80% or 90% w/w of an antibody sample, more usually about 95%, and preferably will be over 99% pure. Antibody purity or homogeneity may be tested by a number of means well known in the art, such as polyacrylamide gel electrophoresis or HPLC.

The term “antagonist antibody” refers to an antibody that binds to a target and prevents or reduces the biological effect of that target. In some embodiments, the term can denote an antibody that prevents the target, e.g., IL-7R, to which it is bound from performing a biological function.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an IL-7R epitope is an antibody that binds this epitope sequence with greater affinity, avidity, more readily, and/or with greater duration than it binds to other sequences. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “immunospecific” binding of antibodies refers to the antigen specific binding interaction that occurs between the antigen-combining site of an antibody and the specific antigen recognized by that antibody (i.e., the antibody reacts with the protein in an ELISA or other immunoassay, and does not react detectably with unrelated proteins).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, the term “IL-7R” refers to any form of IL-7R and variants thereof that retain at least part of the activity of IL-7R. Unless indicated differently, such as by specific reference to human IL-7R, IL-7R includes all mammalian species of native sequence IL-7R, e.g., human, canine, feline, equine, and bovine. One exemplary human IL-7R is found as Uniprot Accession Number P16871 (SEQ ID NO: 1).

(SEQ ID NO: 1)
MTILGTTFGM VFSLLQVVSG ESGYAQNGDL EDAELDDYSF
SCYSQLEVNG SQHSLTCAFE DPDVNTTNLE FEICGALVEV
KCLNFRKLQE IYFIETKKFL LIGKSNICVK VGEKSLTCKK
IDLTTIVKPE APFDLSVIYR EGANDFVVTF NTSHLQKKYV
KVLMHDVAYR QEKDENKWTH VNLSSTKLTL LQRKLQPAAM
YEIKVRSIPD HYFKGFWSEW SPSYYFRTPE INNSSGEMDP
ILLTISILSF FSVALLVILA CVLWKKRIKP IVWPSLPDHK
KTLEHLCKKP RKNLNVSFNP ESFLDCQIHR VDDIQARDEV
EGFLQDTFPQ QLEESEKQRL GGDVQSPNCP SEDVVITPES
FGRDSSLTCL AGNVSACDAP ILSSSRSLDC RESGKNGPHV
YQDLLLSLGT TNSTLPPPFS LQSGILTLNP VAQGQPILTS
LGSNQEEAYV TMSSFYQNQ

Antagonist IL-7R antibodies encompass antibodies that block, antagonize, suppress or reduce (to any degree including significantly) IL-7R biological activity, including downstream pathways mediated by IL-7R signaling, such interaction with IL-7 and/or elicitation of a cellular response to IL-7. For purpose of the present invention, it will be explicitly understood that the term “antagonist IL-7R antibody” (interchangeably termed “IL-7R antagonist antibody,” “antagonist anti-IL-7R antibody” or “anti-IL-7R antagonist antibody”) encompasses all the previously identified terms, titles, and functional states and characteristics whereby the IL-7R itself, an IL-7R biological activity (including but not limited to interaction with IL-7, its ability to mediate any aspect of phosphorylation of STATS, phosphatidylinositol-3-kinase (PI3K)-Akt pathway activation, p27Kip1 downregulation, Bcl-2 upregulation, Rb hyperphosphorylation, and CXCR4 upregulation), or the consequences of the biological activity, are substantially nullified, decreased, or neutralized in any meaningful degree. In some embodiments, an antagonist IL-7R antibody binds IL-7R and prevents interaction with IL-7. Examples of antagonist IL-7R antibodies are provided herein. Anti-IL-7R antagonist antibodies for use in the invention can be identified or characterized using methods known in the art, whereby reduction, amelioration, or neutralization of an IL-7R biological activity is detected and/or measured.

As used herein, the term “C1GM” is used to refer to an antibody comprising the amino acid sequence of the heavy chain and light chain variable regions shown in SEQ ID NO: 2 and SEQ ID NO: 3, respectively.

C1GM heavy chain variable region:
(SEQ ID NO: 2)
EVQLVESGGGLVKPGGSLRLSCAASGFTFDDSVMHWVRQAPGKGLEWVSL
VGWDGFFTYYADSVKGRFTISRDNAKNSLYLQMNSLRAEDTAVYYCARQG
DYMGNNWGQGTLVTVSS
C1GM light chain variable region:
(SEQ ID NO: 3)
NFMLTQPHSVSESPGKTVTISCTRSSGSIDSSYVQWYQQRPGSSPTTVIY
EDDQRPSGVPDRFSGSIDSSSNSASLTISGLKTEDEADYYCQSYDFHHLV
FGGGTKLTVL

The generation and characterization of C1GM is described in the Examples of WO2011/104687, the entire content of which is herein incorporated by reference. In some embodiments, the term “C1GM” refers to immunoglobulin encoded by (a) a polynucleotide encoding C1GM light chain variable region that has a deposit number of ATCC No. PTA-11678, and (b) a polynucleotide encoding C1GM heavy chain variable region that has a deposit number of ATCC No. PTA-11679.

The term “identity” refers to the percent “identity” of two amino acid sequences or of two nucleic acid sequences. The percent identity is generally determined by aligning the sequences for optimal comparison purposes (e.g. gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search that detects distant relationships between molecules (Id.) When utilizing BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

A “therapeutically effective amount” refers to an amount effective, at dosages and for periods of time necessary, to achieve the desired therapeutic result, which in the context of anti-IL-7R antibodies includes treatment or prophylactic prevention of the targeted pathologic condition for example high blood glucose. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Likewise, a therapeutically effective amount of the antibody or antibody portion may vary according to factors such as the disease state, age, sex, and weight of the individual, the ability of the antibody or antibody portion to elicit a desired response in the individual, and the desired route of administration of the antibody formulation. A therapeutically effective amount is also one in which any toxic or detrimental effects of the antibody or antibody portion are outweighed by the therapeutically beneficial effects.

As used herein, the term “treatment” refers to both therapeutic treatment and prophylactic or preventative measures, wherein the object is to prevent or slow down (lessen) the targeted pathologic condition for example high blood glucose. Those in need of treatment include those already with the condition as well as those prone to have the condition or those in whom the condition is to be prevented. As used herein, “treatment” is an approach for obtaining beneficial or desired clinical results including, but not limited to, one or more of the following: including lessening severity, alleviation of one or more symptoms associated with autoimmune disease, including any aspect of autoimmune disease, (such as, for example without limitation, high blood glucose, fever, rash, muscle weakness, etc.).

An “effective amount” of drug, compound, or pharmaceutical composition is an amount sufficient to effect beneficial or desired results including clinical results such as alleviation or reduction of the targeted pathologic condition. An effective amount can be administered in one or more administrations. For purposes of this invention, an effective amount of drug, compound, or pharmaceutical composition is an amount sufficient to treat, ameliorate, reduce the intensity of the targeted pathologic condition. In some embodiments, the “effective amount” may reduce blood glucose levels. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, the term “subject” for purposes of treatment includes any subject, and preferably is a subject who is in need of the treatment of the targeted pathologic condition for example autoimmune disease. For purposes of prevention, the subject is any subject, and preferably is a subject that is at risk for, or is predisposed to, developing the targeted pathologic condition for example autoimmune disease. The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

As used herein, the term “polynucleotide” or “nucleic acid”, used interchangeably herein, means a polymeric form of nucleotides either ribonucleotides or deoxynucleotides or a modified form of either type of nucleotide and may be single and double stranded forms. A “polynucleotide” or a “nucleic acid” sequence encompasses its complement unless otherwise specified. As used herein, the term “isolated polynucleotide” or “isolated nucleic acid” means a polynucleotide of genomic, cDNA, or synthetic origin or some combination thereof, which by virtue of its origin or source of derivation, the isolated polynucleotide has one to three of the following: (1) is not associated with all or a portion of a polynucleotide with which the “isolated polynucleotide” is found in nature, (2) is operably linked to a polynucleotide to which it is not linked in nature, or (3) does not occur in nature as part of a larger sequence.

As used herein, the term “chelating agent” is an excipient that can form at least one bond (e.g., covalent, ionic, or otherwise) to a metal ion. A chelating agent is typically a multidentate ligand that can be used in compositions as a stabilizer to complex with species, which might otherwise promote instability.

As used herein, the term “buffer” refers to an added composition that allows a liquid antibody formulation to resist changes in pH, typically by action of its acid-base conjugate components. When a concentration of a buffer is referred to, it is intended that the recited concentration represent the molar concentration of the free acid or free base form of the buffer.

“Viscosity,” as used herein, may be “absolute viscosity” or “kinematic viscosity.” “Absolute viscosity,” sometimes called dynamic or simple viscosity, is a quantity that describes a fluid's resistance to flow. “Kinematic viscosity” is the quotient of absolute viscosity and fluid density. Kinematic viscosity is frequently reported when characterizing the resistive flow of a fluid using a capillary viscometer. When two fluids of equal volume are placed in identical capillary viscometers and allowed to flow by gravity, a viscous fluid takes longer than a less viscous fluid to flow through the capillary. If one fluid takes 200 seconds to complete its flow and another fluid takes 400 seconds, the second fluid is twice as viscous as the first on a kinematic viscosity scale. If both fluids have equal density, the second fluid is twice as viscous as the first on an absolute viscosity scale. The dimensions of kinematic viscosity are L²/T where L represents length and T represents time. The SI units of kinematic viscosity are m²/s. Commonly, kinematic viscosity is expressed in centistokes, cSt, which is equivalent to mm²/s. The dimensions of absolute viscosity are M/L/T, where M represents mass and L and T represent length and time, respectively. The SI units of absolute viscosity are Pa·s, which is equivalent to kg/m/s. The absolute viscosity is commonly expressed in units of centiPoise, cP, which is equivalent to milliPascal-second, mPa·s.

As used herein, the terms “tonicity agent” or “tonicifier” refers to an excipient that can adjust the osmotic pressure of a liquid antibody formulation. In certain embodiments, the tonicity agent can adjust the osmotic pressure of a liquid antibody formulation to isotonic so that the antibody formulation is physiologically compatible with the cells of the body tissue of the subject. In still other embodiments, the “tonicity agent” may contribute to an improvement in stability of antibodies described herein. An “isotonic” formulation is one that has essentially the same osmotic pressure as human blood. Isotonic formulations generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood, Isotonicity can be measured using a vapor pressure or ice-freezing type osmometer, for example. The tonicity agent can be in an enantiomeric (e.g., L- or D-enantiomer) or racemic form; isomers such as alpha or beta, including alpha, alpha; or beta, beta; or alpha, beta; or beta, alpha; a free acid or free base form; a hydrated form (e.g., monohydrate), or an anhydrous form.

As used herein, the term “polyol” refers an excipient with multiple hydroxyl groups, and includes sugars (reducing and nonreducing sugars), sugar alcohols and sugar acids.

As used herein, the term “surfactant” refers to an excipient that can alter the surface tension of a liquid antibody formulation. In certain embodiments, the surfactant reduces the surface tension of a liquid antibody formulation. In still other embodiments, the “surfactant” may contribute to an improvement in stability of any of the antibody in the formulation. The surfactant may reduce aggregation of the formulated antibody and/or minimize the formation of particulates in the formulation and/or reduces adsorption. The surfactant may also improve stability of the antibody during and after a freeze/thaw cycle.

As used herein, the term “saccharide” refers to a class of molecules that are derivatives of polyhydric alcohols. Saccharides are commonly referred to as carbohydrates and may contain different amounts of sugar (saccharide) units, e.g., monosaccharides, disaccharides and polysaccharides.

As used herein, the term “reducing sugar” is one which contains a hemiacetal group that can reduce metal ions or react covalently with lysine and other amino groups in proteins and a “nonreducing sugar” is one which does not have these properties of a reducing sugar.

A “lyoprotectant” is a molecule which, when combined with a protein of interest, significantly prevents or reduces physicochemical instability of the protein upon lyophilization and subsequent storage. Exemplary lyoprotectants include sugars and their corresponding sugar alcohols; an amino acid such as monosodium glutamate or histidine; a methylamine such as betaine; a lyotropic salt such as magnesium sulfate; a polyol such as trihydric or higher molecular weight sugar alcohols, e.g., glycerin, dextran, erythritol, glycerol, arabitol, xylitol, sorbitol, and mannitol; propylene glycol; polyethylene glycol; Pluronics®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred lyoprotectant are the non-reducing sugars trehalose or sucrose.

The lyoprotectant is added to the pre-lyophilized formulation in a “lyoprotecting amount” which means that, following lyophilization of the protein in the presence of the lyoprotecting amount of the lyoprotectant, the protein essentially retains its physicochemical stability upon lyophilization and storage.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline, normal (0.9%) saline, or 5% dextrose. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “K_{off}”, as used herein, is intended to refer to the off rate constant for dissociation of an antibody from the antibody/antigen complex.

The term “K_{d}”, as used herein, is intended to refer to the dissociation constant of an antibody-antigen interaction. One way of determining the Kd or binding affinity of antibodies to IL-7R is by measuring binding affinity of monofunctional Fab fragments of the antibody. To obtain monofunctional Fab fragments, an antibody (for example, IgG) can be cleaved with papain or expressed recombinantly. The affinity of an anti-IL-7R Fab fragment of an antibody can be determined by surface plasmon resonance (BIAcorC1GM000™ surface plasmon resonance (SPR) system, BIAcore, INC, Piscaway NJ). CM5 chips can be activated with N-ethyl-N′-(3-dimethylaminopropyl)-carbodiimide hydrochloride (EDC) and N-hydroxysuccinimide (NHS) according to the supplier's instructions. Human IL-7R (or any other IL-7R) can be diluted into 10 mM sodium acetate pH 4.0 and injected over the activated chip at a concentration of 0.005 mg/mL. Using variable flow time across the individual chip channels, two ranges of antigen density can be achieved: 100-200 response units (RU) for detailed kinetic studies and 500-600 RU for screening assays. Serial dilutions (0.1-10×estimated KD) of purified Fab samples are injected for 1 min at 100 microliters/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kon) and dissociation rates (koff) are obtained simultaneously by fitting the data to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (KD) values are calculated as k_{off}/kₒₙ. This protocol is suitable for use in determining binding affinity of an antibody to any IL-7R, including human IL-7R, IL-7R of another vertebrate (in some embodiments, mammalian) (such as mouse IL-7R, rat IL-7R, primate IL-7R).

“Reducing incidence” means any of reducing severity (which can include reducing need for and/or amount of (e.g., exposure to) other drugs and/or therapies generally used for this condition. As is understood by those skilled in the art, individuals may vary in terms of their response to treatment, and, as such, for example, a “method of reducing incidence” reflects administering the IL-7R antagonist antibody based on a reasonable expectation that such administration may likely cause such a reduction in incidence in that particular individual.

“Ameliorating” means a lessening or improvement of one or more symptoms as compared to not administering an IL-7R antagonist antibody. “Ameliorating” also includes shortening or reduction in duration of a symptom.

Reference to “about” a value or parameter herein includes (and describes) embodiments that are directed to that value or parameter per se. For example, description referring to “about X” includes description of “X.” Numeric ranges are inclusive of the numbers defining the range.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

When introducing elements of the present invention or the preferred embodiment(s) thereof, the articles “a”, “an”, “the” and “said” are intended to mean that there are one or more of the elements. The terms “comprising”, “comprise”, “comprises”, “including” and “having” are intended to be inclusive and mean that there may be additional elements other than the listed elements. It is understood that wherever embodiments are described herein with the language “comprising,” otherwise analogous embodiments described in terms of “consisting of” and/or “consisting essentially of” are also provided.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the present invention. The materials, methods, and examples are illustrative only and not intended to be limiting.

### Anti-IL-7R Antibody Compositions

In one aspect, the invention provides a formulation comprising an anti-IL-7R antibody, the formulation having viscosity of between about 1 cP and about 20 cP. In another aspect, a method is provided for reducing the viscosity of an anti-IL-7R antibody-containing formulation, wherein the method comprises the step of adding to the formulation a viscosity reducing amount of a compound that is capable of reducing the viscosity of an aqueous formulation comprising said anti-IL-7R antibody. The formulation may be in either aqueous or lyophilized form. In aqueous form, the formulation may have a viscosity of no greater than about 150 cP, preferably no greater than about 120 cP, preferably no greater than about 100 cP, preferably no greater than about 90 cP, preferably no greater than about 80 cP, preferably no greater than about 70 cP, preferably no greater than about 60 cP, preferably no greater than about 50 cP, preferably no greater than about 40 cP, preferably no greater than about 30 cP, preferably no greater than about 20 cP, preferably no greater than about 10 cP, preferably no greater than about 5 cP. In some embodiments the composition comprising antibody has a viscosity of between about 1 cP and about 500 cP, between about 1 cP and 200 cP, between about 1 cP and about 150 cP, between about 1 cP and about 100 cP, between about 1 cP and about 90 cP, between about 1 cP and about 80 cP, between about 1 cP and about 70 cP, between about 1 cP and about 60 cP, between about 1 cP and about 50 cP, between about 1 cP and about 40 cP, between about 1 cP and about 30 cP, between about 1 cP and about 20 cP, or between about 1 cP and about 10 cP at 25° C. In some embodiments, the formulation has a viscosity of about 120 cP, about about 115 cP, 110 cP, about 105 cP, about 100 cP, about 95 cP, about 90 cP, about 85 cP, about 80 cP, about 75 cP, about 70 cP, about 65 cP, about 60 cP, about about 55 cP, 50 cP, about 45 cP, about 40 cP, about 35 cP, about 30 cP, about 25 cP, about 20 cP, about 15 cP, or about 10 cP, or about 5 cP. In some embodiments, the formulation has a viscosity of between about 10 cP and 50 cP, between about 10 cP and 100 cP, between about 20 cP and 60 cP, between about 30 cP and 60 cP, between about 40 cP and 60 cP, or between about 50 cP and 60 cP. In some embodiments, in aqueous form, the formulation may have a viscosity of between about 1 cP and 10 cP. In some embodiments, in aqueous form, the formulation may have a viscosity of between about 1 cP and 15 cP. In some embodiments, in aqueous form, the formulation may have a viscosity of between about 1 cP and 20 cP.

Another aspect of the present invention is directed to an article of manufacture comprising a container holding any of the herein described formulations.

In some embodiments, the formulation comprises at least one anti-IL-7R antibody. In some embodiments, more than one antibody may be present. At least one, at least two, at least three, at least four, at least five, or more, different antibodies can be present. Generally, the two or more different antibodies have complementary activities that do not adversely affect each other. The, or each, antibody can also be used in conjunction with other agents that serve to enhance and/or complement the effectiveness of the antibodies. The antibody may be present in the formulation at a concentration ranging from about 0.1 to about 300 mg/ml. In some embodiments the concentration of antibody is about 0.5 mg/ml, about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 70 mg/ml, about 80 mg/ml, about 90 mg/ml, about 100 mg/ml, about 101 mg/ml, about 102 mg/ml, about 102.5 mg/ml, about 103 mg/ml, about 103.5 mg/ml, about 104 mg/ml, about 104.5 mg/ml, about 105 mg/ml, about 105.5 mg/ml, about 106 mg/ml, about 106.5 mg/ml, about 107 mg/ml, about 107.5 mg/ml, about 108 mg/ml, about 108.5 mg/ml, about 109 mg/ml, about 109.5 mg/ml, about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, about 150 mg/ml, about 151 mg/ml, about 152 mg/ml, about 153 mg/ml, about 154 mg/ml, about 155 mg/ml, about 156 mg/ml, about 157 mg/ml, about 158 mg/ml, about 159 mg/ml, about 160 mg/ml, about 170 mg/ml, about 180 mg/ml, about 190 mg/ml, about 200 mg/ml, about 201 mg/ml, about 202 mg/ml, about 202.5 mg/ml, about 203 mg/ml, about 203.5 mg/ml, about 204 mg/ml, about 204.5 mg/ml, about 205 mg/ml, about 205.5 mg/ml, about 206 mg/ml, about 206.5 mg/ml, about 207 mg/ml, about 207.5 mg/ml, about 208 mg/ml, about 208.5 mg/ml, about 209 mg/ml, about 209.5 mg/ml, about 210 mg/ml, about 211 mg/ml, about 212 mg/ml, about 213 mg/ml, about 214 mg/ml, about 215 mg/ml, about 216 mg/ml, about 217 mg/ml, about 218 mg/ml, about 219 mg/ml, about 220 mg/ml, about 221 mg/ml, about 222 mg/ml, about 223 mg/ml, about 224 mg/ml, about 225 mg/ml, about 226 mg/ml, about 227 mg/ml, about 228 mg/ml, about 229 mg/ml, about 230 mg/ml, about 231 mg/ml, about 232 mg/ml, about 233 mg/ml, about 234 mg/ml, about 235 mg/ml, about 236 mg/ml, about 237 mg/ml, about 238 mg/ml, about 239 mg/ml, about 240 mg/ml, about 241 mg/ml, about 242 mg/ml, about 243 mg/ml, about 244 mg/ml, about 245 mg/ml, about 246 mg/ml, about 247 mg/ml, about 248 mg/ml, about 249 mg/ml, about 250 mg/ml, about 251 mg/ml, about 252 mg/ml, about 253 mg/ml, about 254 mg/ml, about 255 mg/ml, about 256 mg/ml, about 257 mg/ml, about 258 mg/ml, about 259 mg/ml, about 260 mg/ml, about 270 mg/ml, about 280 mg/ml, about 290 mg/ml, or about 300 mg/ml.

According to some embodiments of the present invention the pH can be in the range of about pH 5.0 to 8.0, preferably between about pH 6.5 and of any of about pH 7.0, about 7.1, about 7.2, about 7.3, about 7.4, about 7.5, about 7.6, about 7.7, about 7.8, about 7.9 or about 8.0. Further preferably the pH is in the range selected from between any one of about pH 5.6, 5.7 or 5.8 and any one of about pH 7.5, 7.4, 7.3, 7.2, 7.1, 7.0, 6.9, 6.8, 6.7, 6.6, 6.5, 6.4, 6.3, 6.2, 6.1, 6.0, 5.9, 5.8 or 5.7. In some embodiments the pH can be in the range of between about pH 5.5 and any of about pH 6.0, 6.2, 6.5 or 6.8, alternatively the pH can be in the range of between about pH 5.8 and any of about pH 6.0, 6.2, 6.5 or 6.8. In some embodiments the pH can be selected from pH values of any of about pH 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4 or 7.5, most preferably the pH is pH 7.0+/−0.5. Values of pH in these ranges provide the composition with lower viscosities.

The formulation comprises arginine. In some embodiments, the arginine is arginine hydrochloride, or arginine HCl. The concentration of the arginine can range from about 0.1 millimolar (mM) to about 200 mM. In some embodiments, the concentration of the arginine is from about 10 mM to about 150 mM, about 50 mM to about 130 mM, about 80 mM to about 120 mM, or about 90 mM to about 110 mM. In some embodiments, the concentration of the argnine is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, or about 200 mM.

In some embodiments, the tonicity agent can comprise a polyol, a saccharide, a carbohydrate, a salt, such as sodium chloride, or mixtures thereof. The polyol can have a molecular weight that, for example without limitation, is less than about 600 kD (e.g., in the range from about 120 to about 400 kD), and can be, for example without limitation, mannitol, trehalose, sorbitol, erythritol, isomalt, lactitol, maltitol, xylitol, glycerol, lactitol, propylene glycol, polyethylene glycol, inositol, or mixtures thereof. The saccharide or carbohydrate can be, for example without limitation, a monosaccharide, disaccharide or polysaccharide, or mixtures of any of the foregoing. The saccharide or carbohydrate can be, for example without limitation, fructose, glucose, mannose, sucrose, sorbose, xylose, lactose, maltose, sucrose, dextran, pullulan, dextrin, cyclodextrins, soluble starch, hydroxyethyl starch, water-soluble glucans, or mixtures thereof. The tonicity agent can comprise a saccharide such as, for example without limitation, a reducing sugar or non reducing sugar or mixtures thereof. The tonicity agent can comprise a saccharide which is a non-reducing sugar such as, for example without limitation, sucrose, trehalose, and mixtures thereof.

The concentration of the tonicity agent in the composition ranges from about 1 mg/ml to about 300 mg/ml, from about 1 mg/ml to about 200 mg/ml, or from about 1 mg/ml to about 100 mg/ml. Preferably the concentration of the tonicity agent in the composition is about 0.5 mg/ml, about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 65 mg/ml, about 70 mg/ml, about 75 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 96 mg/ml, about 97 mg/ml, about 98 mg/ml, about 99 mg/ml, about 100 mg/ml, about 101 mg/ml, about 102 mg/ml, about 103 mg/ml, about 104 mg/ml, about 105 mg/ml, about 106 mg/ml, about 107 mg/ml, about 108 mg/ml, about 109 mg/ml, about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, or about 150 mg/ml.

Where the tonicity agent comprises a salt, the concentration of the salt in the composition ranges from about 1 mg/ml to about 20 mg/ml. Salts that are pharmaceutically acceptable and suitable for this invention include sodium chloride, sodium succinate, sodium sulfate, potassium chloride, magnesium chloride, magnesium sulfate, and calcium chloride. In some embodiments the salt in the composition is selected from a range of concentrations of any of about 1 mg/ml, 2 mg/ml, 3 mg/ml, 4 mg/ml, 5 mg/ml, 6 mg/ml, 7 mg/ml, 8, mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml and 20 mg/ml.

The surfactant can be, for example without limitation, a polysorbate, poloxamer, triton, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof. The surfactant can be, for example without limitation, polysorbate 20, polysorbate 21, polysorbate 40, polysorbate 60, polysorbate 61, polysorbate 65, polysorbate 80, polysorbate 81, polysorbate 85, PEG3350 and mixtures thereof.

The concentration of the surfactant generally ranges from about 0.01 mg/ml to about 10 mg/ml, from about 0.01 mg/ml to about 5.0 mg/ml, from about 0.01 mg/ml to about 2.0 mg/ml, from about 0.01 mg/ml to about 1.5 mg/ml, from about 0.01 mg/ml to about 1.0 mg/ml, from about 0.01 mg/ml to about 0.5 mg/ml, from about 0.01 mg/ml to about 0.4 mg/ml, from about 0.01 mg/ml to about 0.3 mg/ml, from about 0.01 mg/ml to about 0.2 mg/ml, from about 0.01 mg/ml to about 0.15 mg/ml, from about 0.01 mg/ml to about 0.1 mg/ml, or from about 0.01 mg/ml, to about 0.05 mg/ml. Further preferably the concentration of the surfactant is about 0.5 mg/ml, about 0.05 mg/ml about 0.06 mg/ml about 0.07 mg/ml about 0.08 mg/ml, about 0.09 mg/ml about 0.1 mg/ml about 0.11 mg/ml about 0.12 mg/ml about 0.13 mg/ml about 0.14 mg/ml about 0.15 mg/ml about 0.16 mg/ml about 0.17 mg/ml about 0.18 mg/ml about 0.19 mg/ml, about 0.2 mg/ml.

The buffer can be, for example without limitation, acetate, succinate, gluconate, citrate, histidine, acetic acid, phosphate, phosphoric acid, ascorbate, tartartic acid, maleic acid, glycine, lactate, lactic acid, ascorbic acid, imidazole, bicarbonate and carbonic acid, succinic acid, sodium benzoate, benzoic acid, gluconate, edetate, acetate, malate, imidazole, tris, phosphate, and mixtures thereof. Preferably the buffer is histidine, wherein the histidine can comprise either L-histidine or D-histidine, a solvated form of histidine, a hydrated form (e.g., monohydrate) of histidine, a salt of histidine (e.g., histidine hydrochloride) or an anhydrous form of histidine or a mixture thereof.

In some embodiments, the buffer, such as for example histidine buffer, provides the composition with a pH close to physiological pH for reduced risk of pain or anaphylactoid side effects on injection and also provides enhanced antibody stability and resistance to aggregation, oxidation and fragmentation.

The concentration of the buffer can range from about 0.1 millimolar (mM) to about 100 mM. Preferably, the concentration of the buffer is from about 0.5 mM to about 50 mM, further preferably about 1 mM to about 30 mM, more preferably about 1 mM to about 18 mM, increasingly preferably about 1 mM to about 15 mM. Preferably, the concentration of the buffer is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 30 mM, about 35 mM, about 40 mM, about 45 mM or about 50 mM. In some embodiments, the concentration of the buffer is about 190 mM, about 200 mM, about 210 mM, about 220 mM, about 230 mM, about 240 mM, about 250 mM, about 260 mM, about 270 mM, about 280 mM, about 290, about 300 mM, about 310 mM, or about 320 mM.

In some embodiments, the chelating agent can be selected from the group consisting of aminopolycarboxylic acids, hydroxyaminocarboxylic acids, N-substituted glycines, 2-(2-amino-2-oxocthyl)aminoethane sulfonic acid (BES), deferoxamine (DEF), citric acid, niacinamide, and desoxycholates and mixtures thereof. In some embodiments, chelating agent is selected from the group consisting of ethylenediaminetetraacetic acid (EDTA), diethylenetriamine pentaacetic acid 5 (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid, N-hydroxyethyliminodiacetic acid (HIMDA), N,N-bis-hydroxyethylglycine (bicine) and N-(trishydroxymethylmethyl) 10 glycine (tricine), glycylglycine, sodium desoxycholate, ethylenediamine; propylenediamine; diethylenetriamine; triethylenetetraamine (trien), ethylenediaminetetraaceto EDTA; disodium EDTA, calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof. In some embodiments, the chelating agent is selected from the group consisting of salts of EDTA including dipotassium edetate, disodium edetate, edetate calcium disodium, sodium edetate, trisodium edetate, and potassium edetate; and a suitable salt of deferoxamine (DEF) is deferoxamine mesylate (DFM), or mixtures thereof. Chelating agents used in the invention can be present, where possible, as the free acid or free base form or salt form of the compound, also as an anhydrous, solvated or hydrated form of the compound or corresponding salt.

Most preferably the chelating agent is either disodium EDTA, calcium EDTA, most preferably disodium EDTA.

Particularly preferable is disodium EDTA as it provides the composition with an enhanced antibody stability and/or resistance to aggregation.

The concentration of chelating agent generally ranges from about 0.01 mg/ml to about 50 mg/ml, from about 1 mg/ml to about 10.0 mg/ml, from about 5 mg/ml to about 15.0 mg/ml, from about 0.01 mg/ml to about 1.0 mg/ml, or from about 0.03 mg/ml to about 0.5 mg/ml. Further preferably concentration of chelating agent generally ranges from from about 0.01 mM to about 2.0 mM, from about 0.01 mM to about 1.5 mM, from about 0.01 mM to about 0.5 mM, from about 0.01 mM to about 0.4 mM, from about 0.01 mM to about 0.3 mM, from about 0.01 mM to about 0.2 mM, from about 0.01 mM to about 0.15 mM, from about 0.01 mM to about 0.1 mM, from about 0.01 mM to about 0.09 mM, from about 0.01 mM to about 0.08 mM, from about 0.01 mM to about 0.07 mM, from about 0.01 mM to about 0.06 mM, from about 0.01 mM to about 0.05 mM, from about 0.01 mM to about 0.04 mM, from about 0.01 mM to about 0.03 mM, from about 0.01 mM to about 0.02 mM or from about 0.05 mM to about 0.01 mM. Preferably the concentration of chelating agent can be about 0.01 mg/ml, 0.02 mg/ml, 0.03 mg/ml, about 0.04 mg/ml, about 0.05 mg/ml, about 0.06 mg/ml, about 0.07 mg/ml, about 0.10 mg/ml, about 0.20 mg/ml. Further preferably the concentration of chelating agent is about 0.045 mg/ml, about 0.046 mg/ml, about 0.047 mg/ml, about 0.048 mg/ml, about 0.049 mg/ml, about 0.05 mg/ml, about 0.051 mg/ml, about 0.052 mg/ml, about 0.053 mg/ml, about 0.054 mg/ml, about 0.055 mg/ml, or about 0.056 mg/ml. Most preferably, the concentration of chelating agent is about 0.05 mg/ml.

Chelating agents can lower the formation of reduced oxygen species, reduce acidic species (e.g., deamidation) formation, reduce antibody aggregation, and/or reduce antibody fragmentation, and/or reduce antibody oxidation in the compositions of the present invention. Such chelating agents can reduce or prevent degradation of an antibody that is formulated in comparison to the antibody without the protection of a chelating agent.

Unless stated otherwise, the concentrations listed herein are those concentrations at ambient conditions, i.e., at 25° C. and atmospheric pressure.

In some embodiments, the formulation can comprise an antioxidant agent. In some embodiments the antioxidant is selected from the group comprising, methionine, sodium thiosulfate, catalase, and platinum.

The concentration of antioxidant generally ranges from about 0.01 mg/ml to about 50 mg/ml, from about 0.01 mg/ml to about 10.0 mg/ml, from about 0.01 mg/ml to about 5.0 mg/ml, from about 0.01 mg/ml to about 1.0 mg/ml, or from about 0.01 mg/ml to about 0.02 mg/ml. Preferably the concentration of antioxidant can be about 0.01 mg/ml, 0.02 mg/ml, 0.03 mg/ml, about 0.04 mg/ml, about 0.05 mg/ml, about 0.06 mg/ml, about 0.07 mg/ml, 0.08 mg/ml, 0.09 mg/ml, about 0.10 mg/ml, 0.11 mg/ml, 0.12 mg/ml, 0.13 mg/ml, about 0.14 mg/ml, about 0.15 mg/ml, about 0.16 mg/ml, about 0.17 mg/ml, 0.18 mg/ml, 0.19 mg/ml about 0.20 mg/ml, about 0.25 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml. Most preferably, the concentration of antioxidant is about 0.01 mg/ml.

In some embodiments the formulation can comprise a preservative. Preferably the preservative agent is selected from Phenol, m-cresol, benzyl alcohol, benzalkonium chloride, benzalthonium chloride, phenoxyethanol and methyl paraben.

The concentration of preservative generally ranges from about 0.001 mg/ml to about 50 mg/ml, from about 0.005 mg/ml to about 15.0 mg/ml, from about 0.008 mg/ml to about 12.0 mg/ml or from about 0.01 mg/ml to about 10.0 mg/ml. Preferably the concentration of preservative can be about 0.1 mg/ml, 0.2 mg/ml, 0.3 mg/ml, about 0.4 mg/ml, about 0.5 mg/ml, about 0.6 mg/ml, about 0.7 mg/ml, 0.8 mg/ml, 0.9 mg/ml about 1.0 mg/ml, 2.0 mg/ml, 3.0 mg/ml, about 4.0 mg/ml, about 5.0 mg/ml, about 6.0 mg/ml, about 7.0 mg/ml, 8.0 mg/ml, 9.0 mg/ml about 9.1 mg/ml, about 9.2 mg/ml, 9.3 mg/ml, 9.4 mg/ml, 9.5 mg/ml, 9.6 mg/ml, 9.7 mg/ml, 9.8 mg/ml, 9.9 mg/ml, 10.0 mg/ml. Most preferably, the concentration of preservative is about 0.1 mg/ml or 9.0 mg/mL.

In some embodiments, the composition does not contain an antioxidant.

In some embodiments, the composition does not contain a preservative.

In some embodiments, the antibody can be selected from the group consisting of monoclonal antibodies, polyclonal antibodies, antibody fragments (e.g., Fab, Fab′, F(ab′)2, Fv, Fc, ScFv etc.), chimeric antibodies, bispecific antibodies, heteroconjugate antibodies, single chain (ScFv), mutants thereof, fusion proteins comprising an antibody portion (e.g., a domain antibody), humanized antibodies, human antibodies, and any other modified configuration of the immunoglobulin molecule that comprises an antigen recognition site of the required specificity, including glycosylation variants of antibodies, amino acid sequence variants of antibodies, and covalently modified antibodies. The antibody may be murine, rat, human, or any other origin (including chimeric or humanized antibodies). In some embodiments, the antibody can be human but is more preferably humanized. Preferably the antibody is isolated, further preferably it is substantially pure. Where the antibody is an antibody fragment this preferably retains the functional characteristics of the original antibody i.e. the ligand binding and/or antagonist or agonist activity.

In some embodiments, the antibody heavy chain constant region may be from any type of constant region, such as IgG, IgM, IgD, IgA, and IgE; and any isotypes, such as IgG1, IgG2, IgG3, and IgG4. Preferably the antibody is an IgG1 or IgG2 antibody.

In some embodiments, the antibody can comprise the human heavy chain IgG2a constant region. In some embodiments the antibody comprises the human light chain kappa constant region. In some embodiments, the antibody comprises a modified constant region, such as a constant region that is immunologically inert, e.g., does not trigger complement mediated lysis, or does not stimulate antibody-dependent cell mediated cytotoxicity (ADCC). In other embodiments, the constant region is modified as described in Eur. J. Immunol. (1999) 29:2613-2624; PCT publication No. WO099/58572; and/or UK Patent Application No. 9809951.8. In still other embodiments, the antibody comprises a human heavy chain IgG2a constant region comprising the following mutations: A330P331 to S330S331 (amino acid numbering with reference to the wildtype IgG2a sequence), Eur. J. Immunol. (1999) 29:2613-2624.

In some embodiments, the antibody is an anti-IL-7R antibody that binds IL-7Rα (such as human IL-7Rα) with a high affinity. In some embodiments, high affinity is (a) binding IL-7R with a K_{D} of less than about 2 nM (such as any of about 1 nM, 800 pM, 600 pM, 400 pM, 200 pM, 100 pM, 90 pM, 80 pM, 70 pM, 60 pM, 50 pM, 40 pM, 30 pM, 20 pM, 10 pM, 5 pM or less).

In some embodiments, antibodies (a) bind IL-7R (such as human IL-7R) with a K_{D} of less than about 2 nM (such as any of about 1 nM, 800 pM, 600 pM, 400 pM, 200 pM, 100 pM, 90 pM, 80 pM, 70 pM, 60 pM, 50 pM, 40 pM, 30 pM, 20 pM, 10 pM, 5 pM or less), and/or a k_{off} of about 4×10⁻⁴ s⁻¹.

The epitope(s) that can be bound by the antibody can be continuous or discontinuous. In one embodiment, the antibody binds essentially the same IL-7R epitope as antibody C1GM.

In some embodiments, the antibody can be anti-IL-7R antibody comprising a heavy chain variable region comprising:
(a) a CDR1 comprising the amino acid sequence shown in SEQ ID NO: 4 (GFTFDDSVMH);

(b) a CDR2 comprising the amino acid sequence shown in SEQ ID NO: 5 (LVGWDGFFTYYADSVKG); and
(c) a CDR3 comprising the amino acid sequence shown in SEQ ID NO: 6 (QGDYMGNN).

In some embodiments, the antibody can be an anti-IL-7R antibody comprising a light chain variable region comprising:

(a) a CDR1 comprising the amino acid sequence shown in SEQ ID NO: 7 (TRSSGSIDSSYVQ);

(b) a CDR2 comprising the amino acid sequence shown in SEQ ID NO: 8 (EDDQRPS); and

(c) a CDR3 comprising the amino acid sequence shown in SEQ ID NO: 9 (QSYDFHHLV).

In some embodiments, the antibody can be anti-IL-7R antibody comprising three CDRs from a heavy chain variable region comprising the amino acid sequence shown in SEQ ID NO: 2.

(SEQ ID NO: 2)
EVQLVESGGGLVKPGGSLRLSCAASGFTFDDSVMHWVRQAPGKGLEWVSL
VGWDGFFTYYADSVKGRFTISRDNAKNSLYLQMNSLRAEDTAVYYCARQG
DYMGNNWGQGTLVTVSS

In some embodiments, the antibody can be anti-IL-7R antibody comprising three CDRs from a light chain variable region comprising the amino acid sequence shown in SEQ ID NO: 3.

(SEQ ID NO: 3)
NFMLTQPHSVSESPGKTVTISCTRSSGSIDSSYVQWYQQRPGSSPTTVIY
EDDQRPSGVPDRFSGSIDSSSNSASLTISGLKTEDEADYYCQSYDFHHLV
FGGGTKLTVL

In some embodiments, the anti-IL-7R antibody may comprise a heavy chain variable region comprising an amino acid sequence of any of at least about 80%, 85%, 90%, 91%, 92%, 93%, 94%, 95%, 96%, 97%, 98% or 99% identical to the amino acid sequence comprising the amino acid sequence shown in SEQ ID NO. 2 and/or a light chain variable region comprising an amino acid sequence of any of at least about 80%, 85%, 90%, 91%, 92%, 93%, 94%, 95%, 96%, 97%, 98% or 99% identical to the amino acid sequence comprising the amino acid sequence shown in SEQ ID NO. 3, wherein the antibody binds specifically to human IL-7Rα.

The anti-IL-7R antibody may comprise a heavy chain variable region comprising the amino acid sequence comprising the amino acid sequence shown in SEQ ID NO: 2 and/or may comprise a light chain variable region comprising the amino acid sequence comprising the amino acid sequence shown in SEQ ID NO: 3.

The anti-IL-7R antibody may be an antibody comprising the amino acid sequences shown in SEQ ID NOS: 2 and 3.

The anti-IL-7R antibody may comprise a heavy chain region comprising an amino acid sequence of any of at least about 80%, 85%, 90%, 91%, 92%, 93%, 94%, 95%, 96%, 97%, 98% or 99% identical to the amino acid sequence comprising the amino acid sequence shown in SEQ ID NO: 10 and/or a light chain region comprising an amino acid sequence of any of at least about 80%, 85%, 90%, 91%, 92%, 93%, 94%, 95%, 96%, 97%, 98% or 99% identical to the amino acid sequence comprising the amino acid sequence shown in SEQ ID NO: 11, wherein the antibody binds specifically to human IL-7Rα.

Heavy chain region sequence
(SEQ ID NO: 10)
EVQLVESGGGLVKPGGSLRLSCAASGFTFDDSVMHWVRQAPGKGLEWVSL
VGWDGFFTYYADSVKGRFTISRDNAKNSLYLQMNSLRAEDTAVYYCARQG
DYMGNNWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK
Light chain region sequence
(SEQ ID NO: 11)
NFMLTQPHSVSESPGKTVTISCTRSSGSIDSSYVQWYQQRPGSSPTTVIY
EDDQRPSGVPDRFSGSIDSSSNSASLTISGLKTEDEADYYCQSYDFHHLV
FGGGTKLTVLQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS

The anti-IL-7R antibody may comprise a heavy chain region comprising the amino acid sequence comprising the amino acid sequence shown in SEQ ID NO: 10 and/or may comprise a light chain region comprising the amino acid sequence comprising the amino acid sequence shown in SEQ ID NO: 11.

The anti-IL-7R antibody may be an antibody comprising the amino acid sequences shown in SEQ ID NOS: 10 and 11.

The anti-IL-7R antibody may compete for IL-7R binding with an anti-IL-7R antibody as defined herein. The anti-IL-7R antibody may compete for IL-7R binding with an antibody comprising a heavy chain variable region comprising the amino acid sequence comprising the amino acid sequence shown in SEQ ID NO: 2 and/or a light chain variable region comprising the amino acid sequence comprising the amino acid sequence shown in SEQ ID NO: 3.

The anti-IL-7R antibody may be a human and affinity matured antibody, C1GM, which specifically binds human IL-7Rα. Antibody C1GM is described in WO2011/104687, the content of which is hereby incorporated by reference in its entirety. The amino acid sequences of the heavy chain and light chain variable regions of C1GM are shown in SEQ ID NOs: 2 and 3, respectively. The CDR portions of antibody C1GM (including Chothia and Kabat CDRs) are diagrammatically depicted in Table 1 of WO2011/104687. Antibody C1GM is highly potent in blocking IL-7R biological activity.

The anti-IL-7R antibody may also comprise a fragment or a region of the antibody C1GM. In one embodiment, the fragment is a light chain of the antibody C1GM comprising the amino acid sequence as shown in SEQ ID NO: 11 herein. In another embodiment, the fragment is a heavy chain of the antibody C1GM comprising the amino acid sequence as shown in SEQ ID NO: 10 herein. In yet another embodiment, the fragment contains one or more variable regions from a light chain and/or a heavy chain of the antibody C1GM. In yet another embodiment, the fragment contains one or more CDRs from a light chain and/or a heavy chain of the antibody C1GM comprising the amino acid sequences as shown in SEQ ID NOS: 11 and 10, respectively, herein.

In some embodiments, the antibody may comprise any one or more of the following: a) one or more (one, two, three, four, five, or six) CDR(s) derived from antibody C1GM shown in SEQ ID NOs: 1-6. In some embodiments, the CDRs may be Kabat CDRs, Chothia CDRs, or a combination of Kabat and Chothia CDRs (termed “extended” or “combined” CDRs herein). In some embodiments, the polypeptides comprise any of the CDR configurations (including combinations, variants, etc.) described herein.

In some embodiments of the present invention the C-terminal lysine of the heavy chain of any of the anti-IL-7R antibodies described herein is deleted. In various cases the heavy and/or light chain of the anti-IL-7R antibodies described herein may optionally include a signal sequence.

In another embodiment, the antibody may be selected from an anti-IL-7R antibody known in the art, such as antibodies described in, for example without limitation, any of the following published PCT applications: WO2011/104687 (including, for example without limitation, any of the antibodies listed in Table 1), WO/2011/094259 (including, for example without limitation, antibodies H3L4, BPC4401, BPC4398, BPC1142, BPC4399, BPC4402, BPC4403, and BPC1142), WO/2013/056984 (including, for example without limitation, antibodies MD707-1, MD707-2, MD707-3, MD707-4, MD707-5, MD707-6, MD707-9, MD707-12, and MD707-13), and WO2010/017468 (including, for example without limitation, antibodies 9B7, R34.34, 6A3 and 1A11). The antibody may bind to the same epitope as an anti-IL-7R antibody known in the art and/or may compete for binding to IL-7R with such an antibody.

According to a further aspect of the present invention there is provided a composition comprising or consisting of;

about 100 mg/ml to about 150 mg/ml of antibody,

about 10.0 mM to about 30.0 mM histidine buffer,

about 1 mg/ml to about 100 mg/ml sucrose,

about 0.01 to about 0.3 mg/ml polysorbate 80 (PS80),

about 0.01 to about 0.1 mg/ml disodium EDTA,

about 50 mM to about 150 mM arginine HCl,

wherein said composition is of a pH selected from the the range of between about pH 6.0 and any of about pH 7.0, 7.5, or 8.0, or alternatively from the range of between about pH 6.0 and any of about pH 6.5, 6.6, 6.7, 6.8, 7.0, 7.1, 7.2, 7.3, 7.4, 7.5, 7.6, 7.7, 7.8, 7.9, or 8.0.

According to a further aspect of the present invention there is provided a composition comprising or consisting of any of about 90 mg/ml, about 100 mg/ml, about 110 mg/ml, about 120 mg/ml, about 130 mg/ml, about 140 mg/ml or about 150 mg/ml of antibody,

about 10.0 mM to about 30.0 mM histidine buffer,

about 1 mg/ml to about 100 mg/ml sucrose,

about 0.01 to about 0.3 mg/ml PS80,

about 0.01 to about 0.1 mg/ml disodium EDTA,

about 50 mM to about 150 mM arginine HCl or NaCl,

wherein said composition is of a pH selected from the the range of between about pH 5.8 and any of about pH 5.8, 5.9, 6.0, 6.1, 6.2, 6.3, 6.4, 6.5 6.6, 6.7, 6.8, 6.9, 7.0, 7.1, 7.2, 7.3, 7.4, or 7.5, or alternatively from the range of between about pH 6.5 and any of about pH 6.5, 6.8, 7.0, 7.1, 7.2, 7.3, 7.4, or 7.5.

According to a preferred embodiment the composition comprises or consists of any of about 90 mg/ml, about 100 mg/ml, about 110 mg/ml, about 120 mg/ml, about 130 mg/ml, about 140 mg/ml or about 50 mg/ml of antibody,

about 20 mM histidine buffer,

about 50 mg/ml sucrose,

about 0.2 mg/ml PS80,

about 0.05 mg/ml disodium EDTA,

about 100 mM arginine HCl or NaCl

wherein said composition is of a pH selected from the the range of between about pH 6.0 and any of about pH 6.0, 6.2, 6.5 or 6.8, or alternatively from the range of between about pH 6.5 and any of about pH 6.5, 6.8, 7.0, 7.1, 7.2, 7.3, 7.4, or 7.5, and wherein said antibody comprises a variable heavy chain sequence comprising the amino acid sequence shown in SEQ ID NO. 1 and a variable light chain sequence comprising the amino acid sequence shown in SEQ ID NO. 2.

According to a preferred embodiment the composition comprises or consists of any of about 90 mg/ml, about 100 mg/ml, about 110 mg/ml, about 120 mg/ml, about 130 mg/ml, about 140 mg/ml or about 150 mg/ml of antibody,

about 20 mM histidine buffer,

about 50 mg/ml sucrose,

about 0.2 mg/ml PS80,

about 0.05 mg/ml disodium EDTA,

about 100 mM arginine HCl or NaCl,

wherein the pH of said composition is about pH 7.0, +/−0.5 and wherein said antibody comprises a variable heavy chain sequence comprising the amino acid sequence shown in SEQ ID NO. 1 and a variable light chain sequence comprising the amino acid sequence shown in SEQ ID NO. 2. In some embodiment the dose volume used is about 0.5 ml, about 1 ml, about 2 ml, about 3 ml, about 4 ml, about 5 ml, about 6 ml, about 7 ml, about 8 ml, about 9 ml, about 10 ml, about 11 ml, about 12 ml, about 13 ml, about 14 ml, about 15 ml, about 16 ml, about 17 ml, about 18 ml, about 19 ml, about 20 ml, about 21 ml, about 22 ml, about 23 ml, about 24 ml, about 25 ml, about 26 ml, about 27 ml, about 28 ml, about 29 ml, about 30 ml, about 31 ml, about 32 ml, about 33 ml, about 34 ml, about 35 ml, about 36 ml, about 37 ml, about 38 ml, about 39 ml, about 40 ml, about 41 ml, about 42 ml, about 43 ml, about 44 ml, about 45 ml, about 46 ml, about 47 ml, about 48 ml, about 49 ml, or about 50 ml.

In some embodiments there is provided a composition which is lyophilized and/or has been subjected to lyophylization. In some embodiments there is provided a composition which is not lyophilized and has not been subjected to lyophylization.

In some embodiments the concentration of antibody is any of about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, or about 160 mg/ml.

According to a further preferred aspect of the present invention there is provided a composition, according to any foregoing aspect or embodiment, for the manufacture of a medicament for treatment of an autoimmune disease or type 2 diabetes in a mammal.

In some embodiments, the autoimmune disorder is selected from one or more of type 1 diabetes, rheumatoid arthritis, lupus, multiple sclerosis, and GVHD.

According to a yet further embodiment of the invention there is provided a composition, according to any foregoing aspect or embodiment, for the manufacture of a medicament for treatment of autoimmune disease or type 2 diabetes.

According to a yet further embodiment of the invention there is provided a composition, according to any foregoing aspect or embodiment, for the manufacture of a medicament for treatment of autoimmune disease or type 2 diabetes. According to another aspect there is provided a composition, according to any foregoing aspect or embodiment, for the manufacture of a medicament for treatment of autoimmune disease or type 2 diabetes.

Preferably the mammal is selected from rodents (such as mice, rats and rabbits, pets (such as cats, dogs and horses), farm animals (such as cows, sheep, pigs and goats), sport animals and/or pets (such as cats, dogs and horses), primates, more preferably a human.

According to a preferred embodiment the composition can be administered directly into the blood stream, into muscle, into tissue, into fat, or into an internal organ. Suitable means for parenteral administration include intravenous, intraarterial, intraperitoneal, intrathecal, intraventricular, intraurethral, intrasternal, intracranial, intramuscular, intra-ossial, intradermal and subcutaneous. Suitable devices for parenteral administration include needle (including microneedle, microprojections, soluble needles and other micropore formation techniques) injectors, needle-free injectors and infusion techniques.

In some embodiments the administration pattern of the medicament comprises administration of a dose of the medicament once every week, once every two weeks, once every three weeks, once every four weeks, once every five weeks, once every six weeks, once every seven weeks, once every eight weeks, once every nine weeks, once every ten weeks, once every fifteen weeks, once every twenty weeks, once every twenty five weeks, or once every twenty six weeks. In some embodiments, the anti-IL-7R antagonist antibody is administered once every month, once every two months, once every three months, once every four months, once every five months, or once every six months. In some embodiments the administration pattern of the medicament comprises administration of a dose of the medicament once every four or eight weeks.

In some embodiments the volume of a dose is less than or equal to about 20 ml, about 15 ml, about 10 ml, about 5 ml, about 2.5 ml, about 1.5 ml, about 1.0 ml, about 0.75 ml, about 0.5 ml, about 0.25 ml or about 0.01 ml.

In some embodiments the volume of a dose is about 20 ml, about 19 ml, about 18 ml, about 17 ml, about 16 ml, about 15 ml, about 14 ml, about 13 ml, about 12 ml, about 11 ml, about 10 ml, about 9 ml, about 8 ml, about 7 ml, about 6 ml, about 5 ml, about 4 ml, about 3 ml, about 2 ml or about 1 ml. Alternatively about 20.5 ml, about 19.5 ml, about 18.5 ml, about 17.5 ml, about 16.5 ml, about 15.5 ml, about 14.5 ml, about 13.5 ml, about 12.5 ml, about 11.5 ml, about 10.5 ml, about 9.5 ml, about 8.5 ml, about 7.5 ml, about 6.5 ml, about 5.5 ml, about 4.5 ml, about 3.5 ml, about 2.5 ml, about 1.5 ml, or about 0.5. Alternatively about 900 microliters, about 800 microliters, about 700 microliters, about 600 microliters, about 500 microliters, about 400 microliters, about 300 microliters, about 200 microliters, or about 100 microliters, alternatively about 950 microliters, about 850 microliters, about 750 microliters, about 650 microliters, about 550 microliters, about 450 microliters, about 350 microliters, about 250 microliters, about 150 microliters, or about 50 microliters. In some embodiments the volume of the dose is less than or equal to about 1.0 ml.

According to preferred embodiment the concentration of antibody can range from about 0.1 to about 200 mg/ml. Preferably the concentration of antibody is about 0.5 mg/ml, about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 61 mg/ml, about 62 mg/ml, about 63 mg/ml, about 64 mg/ml, about 65 mg/ml, about 66 mg/ml, about 67 mg/ml, about 68 mg/ml, about 69 mg/ml, about 70 mg/ml, about 71 mg/ml, about 72 mg/ml, about 73 mg/ml, about 74 mg/ml, about 75 mg/ml, about 76 mg/ml, about 77 mg/ml, about 78 mg/ml, about 79 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 96 mg/ml, about 97 mg/ml, about 98 mg/ml, about 99 mg/ml, about 100 mg/ml, about 101 mg/ml, about 102 mg/ml, about 103 mg/ml, about 104 mg/ml, about 105 mg/ml, about 106 mg/ml, about 107 mg/ml, about 108 mg/ml, about 109 mg/ml, or about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, or about 150 mg/ml. Most preferably the concentration of antibody is less than or equal to 120 mg/ml and may be selected from the group comprising about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, or about 150 mg/ml.

According to a preferred embodiment the dose contains less than or equal to about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 850 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg of antibody.

According to a preferred embodiment the dose contains an amount of antibody that is about 1 μg/kg, about 10 μg/kg, about 20 μg/kg, about 25 μg/kg, about 50 μg/kg, about 100 μg/kg, about 200 μg/kg, about 250 μg/kg, about 500 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or about 11 mg/kg (of mass of the mammal to which the dose it to be administered). In some embodiments, the dose contains about 20 μg/kg, about 25 μg/kg, about 50 μg/kg, about 100 μg/kg, about 200 μg/kg, about 250 μg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg.

Dosage regimens may depend on the pattern of pharmacokinetic decay that the practitioner wishes to achieve. For example, in some embodiments, dosing from one-four times a week is contemplated. Even less frequent dosing may be used. In some embodiments, the dose is administered once every 1 week, every 2 weeks, every 3 weeks, every 4 weeks, every 5 weeks, every 6 weeks, every 7 weeks, every 8 weeks, every 9 weeks, every 10 weeks, every 15 weeks, every 20 weeks, every 25 weeks, or longer. In some embodiments, the dose is administered once every 1 month, every 2 months, every 3 months, every 4 months, every 5 months, every 6 months, or longer. The progress of this therapy is easily monitored by conventional techniques and assays. The dosing regimen can vary over time.

For the purpose of the present invention, the appropriate dosage of the medicament will depend on the antibody employed, the type and severity of the disorder to be treated, whether the agent is administered for preventative or therapeutic purposes, previous therapy, the patient's clinical history and response to the agent, and the discretion of the attending physician. Typically the clinician will administer the medicament, until a dosage is reached that achieves the desired result. Dosages may be determined empirically. For example individuals are given incremental dosages to assess efficacy of the medicament, blood glucose levels may be followed.

Dose and/or frequency can vary over course of treatment. Empirical considerations, such as the antibody half-life, generally will contribute to the determination of the dosage. Frequency of administration may be determined and adjusted over the course of therapy, and is generally, but not necessarily, based on treatment and/or suppression and/or amelioration and/or delay of one or more symptoms of autoimmune disease. In some individuals, more than one dose may be required. Frequency of administration may be determined and adjusted over the course of therapy. For example without limitation, for repeated administrations over several days or longer, depending on the disease and its severity, the treatment is sustained until a desired suppression of symptoms occurs or until sufficient therapeutic levels are achieved to reduce blood glucose levels.

Administration of medicament comprising the composition can be continuous or intermittent, depending, for example, upon the recipient's physiological condition, whether the purpose of the administration is therapeutic or prophylactic, and other factors known to skilled practitioners. The administration of the medicament comprising the composition may be essentially continuous over a preselected period of time or may be in a series of spaced dose.

Preferably the administration of the dose is a parenteral administration preferably selected from intravenous, intraarterial, intraperitoneal, intrathecal, intraventricular, intraurethral, intrasternal, intracranial, intramuscular, intra-ossial, intradermal and subcutaneous. Preferably the medicament is in a unit dosage sterile form for parenteral administration.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims. The Examples in WO2011/104687 are referred to illustrate the antibodies for use in the present invention. The entire content of WO2011/104687 is hereby incorporated by reference.

### EXAMPLES

### Example 1. Anti-IL-7R Antibody Formulation 1

This example illustrates the viscosity of high concentration anti-IL-7R antibody formulations.

Formulation 1 was amenable to achieve concentrations of approximately 50-70 mg/mL C1GM antibody (in 20 mM histidine, 85 g/L sucrose, 0.05 g/L disodium EDTA dihydrate, 0.2 g/L polysorbate-80, pH 5.8), with suitable stability characteristics. The antibody has also shown opalescence in this formulation, a phenomenon which is not related to particle formation.

Studies were conducted to evaluate impact of pH change (below and above isoelectric point, pl). The drug product was formulated as a lyophilized powder for reconstitution with sWFI (Table 1). Viscosity was evaluated using an Anton-Paar rheometer in cone-plate configuration, at 25° C. The sample size was approximately 81 uL. The samples were measured with a constant shear rate (898 s-1).

TABLE 1

| Component | Formulation 1 | |
|---|---|---|
| Antibody C1GM | 0-200 | mg/mL |
| L-histidine | 20 | mM |
| Sucrose | 85 | g/L |
| Disodium EDTA | 0.05 | g/L |
| Polysorbate 80 | 0.2 | g/L |
| Arginine HCl | n/a | |
| WFI | q.s. 1.0 | mL |

| | |
|---|---|
| pH | 5.0 ± 0.5 |
| Presentation | lyophilized, 100 mg/vial (=2 mL |
| | solution after reconstitution) |

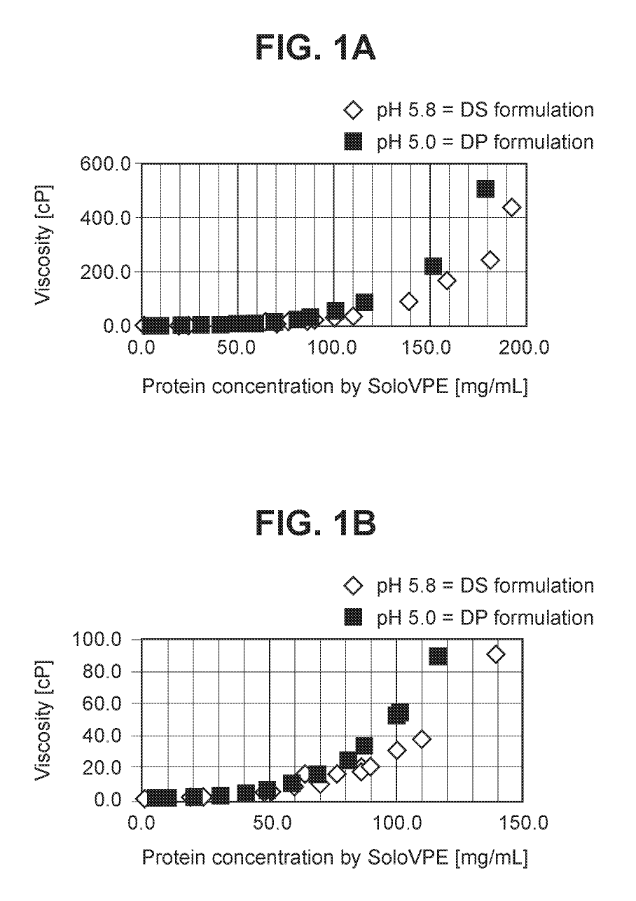

A pH adjustment to pH 5.0 for the drug product resulted in acceptable opalescence values. High viscosity was observed at both pH 5.0 and 5.8 (FIGS. 1A and 1B: viscosity of formulation at pH 5.8 and pH 5.0 (A) up to approximately 200 mg/mL C1GM; (B) y-axis scale limited to 100 cP).

These results demonstrate that lowering pH resulted in acceptable opalescence values.

### Example 2. Anti-IL-7R Antibody with Arginine HCl

This example illustrates the impact of arginine HCl on viscosity in a new anti-IL-7R antibody formulation, formulation 2.

A study was conducted to assess the viscosity of formulation 2. Formulation 2, shown in the right-hand column of Table 2 below, includes 100 mM arginine HCl.

TABLE 2

| Component | Formulation 1 | | Formulation 2 | |
|---|---|---|---|---|
| Antibody C1GM | 101.4 to 179.1 | mg/mL | 101.8 to 182.6 | mg/mL |
| L-histidine | 20 | mM | 20 | mM |
| Sucrose | 85 | g/L | 50 | g/L |
| Disodium EDTA | 0.05 | g/L | 0.05 | g/L |
| Polysorbate 80 | 0.2 | g/L | 0.2 | g/L |
| Arginine HCl | n/a | | 100 | mM |
| WFI | q.s. 1.0 | mL | q.s. 1.0 | mL |

| | | |
|---|---|---|
| pH | 5.0 ± 0.5 | 7.0 ± 0.5 |
| Presentation | lyophilized, 100 mg/vial | Liquid or lyophilized |
| | (=2 mL solution | |
| | after reconstitution) | |

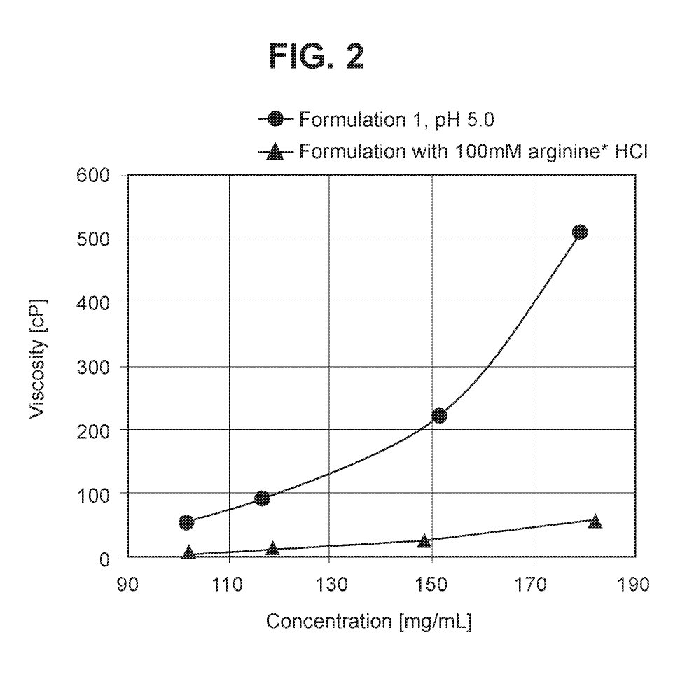

Viscosity was evaluated using an Anton-Paar rheometer in cone-plate configuration, at 25° C. The sample size was approximately 81 uL. The samples were measured with a constant shear rate (898 s-1). Viscosity data are summarized in Table 3 below and FIG. 2.

TABLE 3

| | Antibody | |
|---|---|---|
| | concentration | Viscosity at 25° C. |
| Formulation | (mg/mL) | (cP) |
| Formulation 1, | 179.1 | 506.3 |
| pH 5 | 151.8 | 221.8 |
| | 116.1 | 89.5 |
| | 101.4 | 55.1 |
| Formulation 2 with 100 mM | 182.6 | 55.1 |
| arginine HCl, pH 7.0 | 148.3 | 25.7 |
| | 118.8 | 9.7 |
| | 101.8 | 5.5 |

Viscosity of formulation 2 containing 100 mM arginine HCl showed significantly reduced viscosity, i.e., approximately 10-fold reduction in viscosity, compared to formulation 1 at all antibody concentrations tested (Table 3 and FIG. 2). For example, at 118.8 mg/ml antibody, viscosity of formulation 2 was 9.7 cP, compared viscosity of formulation 1 at 116.1 mg/ml antibody, which was 89.5 cP. At about 101 mg/ml antibody, viscosity of formulation 2 was 5.5 cP, compared to viscosity of formulation 1, which was 55.1 cP. At about 150 mg/ml antibody, viscosity of formulation 2 was 25.7 cP, compared to viscosity of formulation 1, which was 221.8 cP. At about 180 mg/ml antibody, viscosity of formulation 2 was 55.1 cP, compared to viscosity of formulation 1, which was 506.3 cP.

These results demonstrate the inclusion of arginine HCl significantly reduces viscosity of an anti-IL-7R antibody formulation. Formulation 2, which contains 100 mM arginine hydrochloride and has pH 7, allows C1GM protein concentrations of greater than 100 mg/mL with viscosity behavior suitable for use in therapeutic treatment. This was not possible for C1GM in formulation 1 because of high viscosity. Formulation 2 has a target concentration of 120 mg/mL, a 2.4× increase in concentration compared to formulation 1, with a viscosity that is below 20 cP. Feasibility of a lyophilized format of this formulation has been shown. The manufacturability of material at approximately 130 mg/mL in this formulation has been demonstrated in a pilot scale process run using a 500 L bioreactor.

### Example 3. Impact of pH on Viscosity

This example illustrates the impact of pH on viscosity in an anti-IL-7R antibody formulation.

A study was conducted to evaluate the impact of pH on formulation 1. C1GM formulated drug was dialyzed into pH 4.0 glutamate, pH 5.0 histidine, pH 5.8 histidine (at 20 mM buffer concentration), using laboratory scale cassettes. After concentration (in centricons with molecular weight cutoff of 30 kDa), the actual pH values were pH 4.6, 5.2, and 5.8. The pH 4.6 glutamate sample was titrated with 0.1N HCl to achieve pH 4.0.

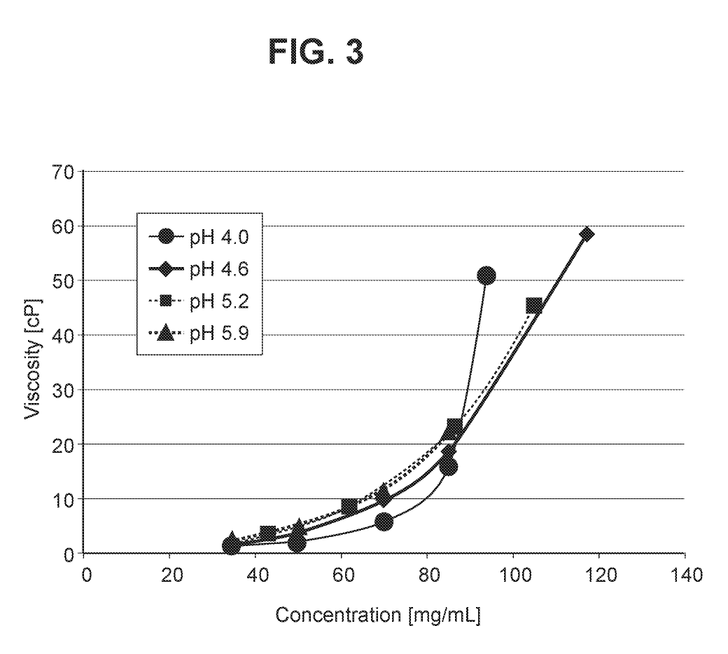

Viscosity was evaluated using an Anton-Paar rheometer in cone-plate configuration, at 25° C. The sample size was approximately 81 uL. The samples were measured with a constant shear rate (898 s-1). Results are summarized in FIG. 3.

Viscosities of anti-IL-7R formulation at pH 5.9, 5.2 and 4.6 were not significantly different (FIG. 3). Viscosity at pH 4.0 showed an increase at 90 mg/ml antibody.

These results demonstrate that pH adjustment to lower values did not show significant impact on viscosity, and low pH preparations indicate a trend to higher viscosity at concentrations above 90 mg/mL, compared to the formulation at pH 5.8.

### Example 4. Impact of Added Excipients on Viscosity

This example illustrates the impact of sodium chloride and arginine HCl on viscosity in an anti-IL-7R antibody formulation.

Stock solutions of arginine hydrochloride and sodium chloride were prepared in the respective buffers at a concentration of 0.75 M arginine HCl or 1M NaCl. Low volume spikes were added to the buffered protein solutions to achieve a final excipient concentration of 150 mM.

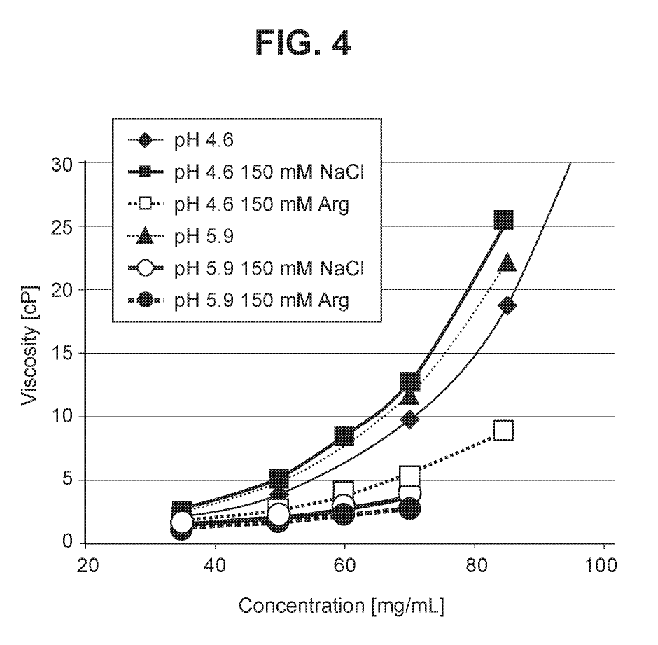

Viscosity at pH 4.6 and pH 5.9 with 150 mM excipient (NaCl or arginine HCl) was evaluated using an Anton-Paar rheometer in cone-plate configuration, at 25° C. The sample size was approximately 81 uL. The samples were measured with a constant shear rate (898 s-1). Results are summarized in FIG. 4.

Significant decrease in viscosity was achieved when adding NaCl or arginine HCl to anti-IL-7R antibody formulation 1 at pH 5.9 (FIG. 4). For example, at pH 5.9 and 70 mg/ml antibody, viscosity of antibody formulation without added excipient was about 12 cP, viscosity of antibody formulation with 150 mM NaCl was about 4 cP, viscosity of antibody formulation with 150 mM arginine HCl was about 3 cP. Arginine HCl addition was seen to have an effect at lower pH as well.

These results demonstrate that addition of arginine HCl or NaCl significantly reduces viscosity of an anti-IL-7R antibody formulation.

### Example 5. Impact of pH on Viscosity

This example illustrates the impact of sample preparation at higher pH on viscosity in an anti-IL-7R antibody formulation.

Antibody C1GM has a calculated pl of 6.8. Since previous studies indicated low pH had little or negative impact on viscosity, samples were prepared at higher pH using the following buffers:

a. 20 mM Histidine, pH 7.0

b. 20 mM Histidine, 150 mM NaCl, pH 7.0

c. 20 mM Histidine, 150 mM Arginine HCl, pH 7.0

d. 20 mM Tris, pH 8.0

e. 20 mM Tris, 150 mM NaCl, pH 8.0

0.5 mL samples were buffer exchanged in centricons.

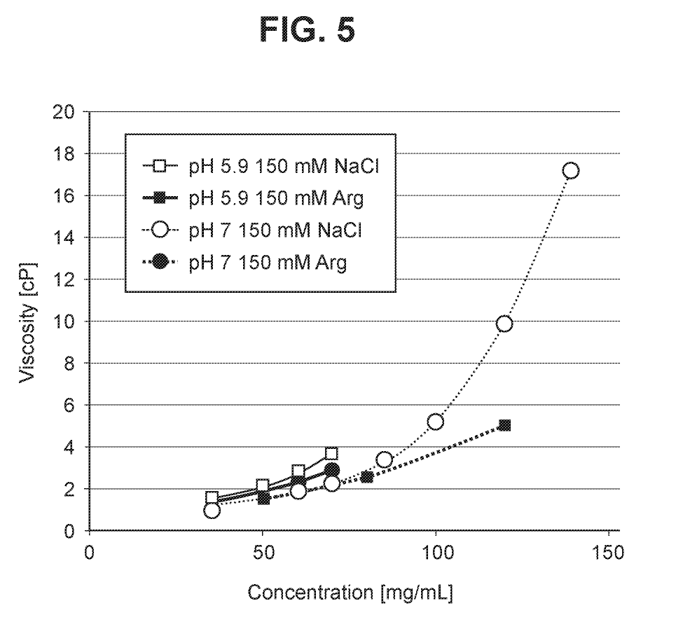

Viscosity at pH 7 with 150 mM excipient (NaCl or arginine HCl) was evaluated using an Anton-Paar rheometer in cone-plate configuration, at 25° C. The sample size was approximately 81 uL. The samples were measured with a constant shear rate (898 s-1). Results are summarized in FIG. 5.

Samples at pH 7 and pH 8 with no salt showed phase separation. However, in samples with the addition of sodium chloride and arginine HCl at 150 mM, a further decrease in viscosity could was observed at pH 7 compared to pH 5.9 (FIG. 5). Additional increase to pH 8 and a change in the buffer had limited effect (data not shown). At pH 7, in the concentration range above approximately 80 mg/mL of anti-IL-7R antibody, arginine HCl addition provided formulations with lower viscosity than sodium chloride addition (FIG. 5, 150 mM arginine HCl (closed squares) compared to 150 mM NaCl (open circles)).

These results demonstrate that an arginine-containing anti-IL-7R antibody formulation at pH 7 has lower viscosity than a sodium chloride-containing formulation.

### Example 6. Impact of Excipient Concentration on Viscosity

This example illustrates the impact of varying excipient concentration on viscosity in an anti-IL-7R antibody formulation.

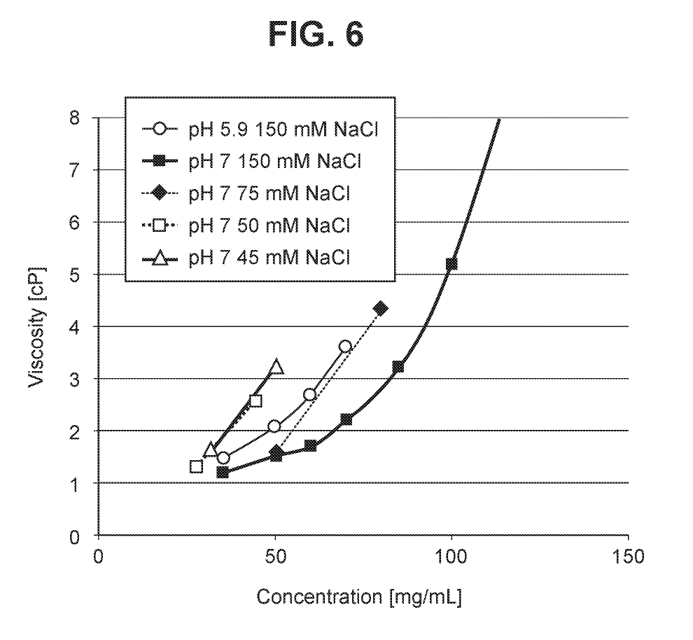

Excipient concentrations were reduced by diluting C1GM samples containing 150 mM sodium chloride with 20 mM histidine buffer, pH 7, to obtain viscosities of formulations with 45, 50, 75 mM sodium chloride. Viscosities at pH 5.9 or pH 7 with 45, 50, 75, 150 mM NaCl was evaluated using an Anton-Paar rheometer in cone-plate configuration, at 25° C. The sample size was approximately 81 uL. The samples were measured with a constant shear rate (898 s-1). Results are summarized in FIG. 6.

Lower amounts of sodium chloride led to higher viscosities than observed for the higher amount of 150 mM sodium chloride (FIG. 6). Phase separation was observed at pH 7 in solutions with low ionic strength (i.e. no salt addition).

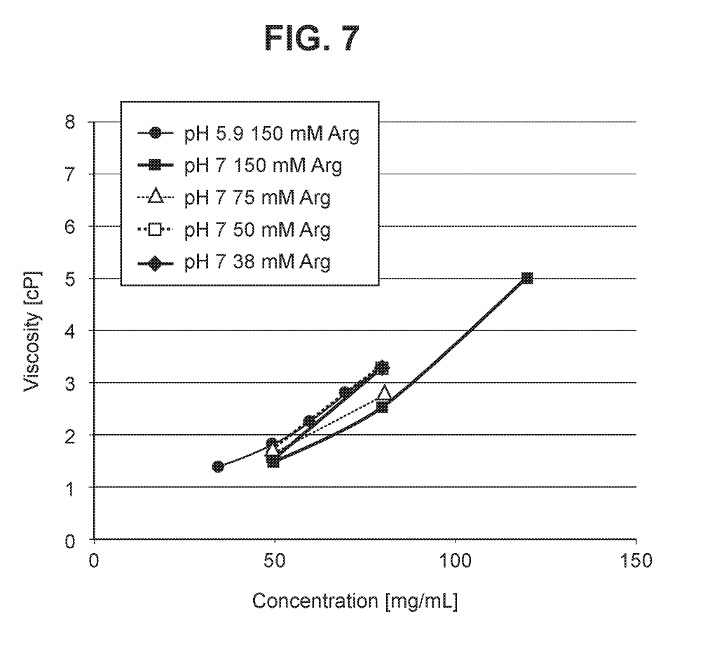

Excipient concentrations were reduced by diluting C1GM samples containing 150 mM arginine hydrochloride with 20 mM histidine buffer, pH 7, to obtain viscosities of formulations with 38, 50, 75 mM arginine hydrochloride. Viscosities at pH 5.9 or pH 7 with 38, 50, 75, 150 mM arginine HCl were evaluated using an Anton-Paar rheometer in cone-plate configuration, at 25° C. The sample size was approximately 81 uL. The samples were measured with a constant shear rate (898 s-1). Results are summarized in FIG. 7.

Lower amounts of excipient had less effect on viscosity. Phase separation has been observed at pH 7 in solutions with low ionic strength (i.e. no salt addition). The concentration effect of arginine HCl appeared less pronounced than for sodium chloride.

These results demonstrate that arginine HCl addition appears to provide some robust protection against viscosity increases over the range of the ionic strength of the formulation.

### Example 7. Short-Term Stability Assessment of Anti-IL-7R Antibody Formulation

This example illustrates the stability assessment of an anti-IL-7R antibody formulation.

For robustness against stressors such as freezing, agitation, and elevated temperature, protein formulations generally require excipients in addition to the buffer. Sucrose was selected as the stabilizing disaccharide for formulations 1 and 2. Disodium EDTA (chelating agent) and polysorbate-80 (PS80, surfactant) were selected as stabilizers for formulations 1 and 2.

Osmolality of the formulation is an important consideration for a suitable drug product for therapeutic use. Stabilizing excipients such as sucrose contribute to the tonicity of the formulation.

The osmolality of a 20 mM histidine formulation with 150 mM excipient alone was calculated to be above approximately 400 mOsm/kg. In order to stay close to the isotonic range (approx. 280-320 mOsm/kg) and to allow addition of the necessary amount of sucrose, the concentration of the viscosity lowering excipient (sodium chloride, or arginine hydrochloride) was selected at 100 mM.

To assess short-term stability of the anti-IL-7R antibody formulations, formulations were prepared at 150 mg/mL C1GM antibody by use of dialysis and concentrators (in centricons with molecular weight cutoff of 30 kDa), and spike of concentrated arginine hydrochloride or sodium chloride solutions, respectively. Samples were subsequently placed on short-term stability (8 weeks at 40° C. and 5° C.). Protein stability was assessed with regard to aggregation (by SEC-HPLC), fragmentation (capillary electrophoresis), charge isoforms (iCE), concentration (A280) and pH. The control formulation was formulation 1 at pH 5 (see Example 1 above), concentrated to 150 mg/mL.

Viscosities of anti-IL-7R antibody C1GM formulation (20 mM histidine, 50 g/L sucrose, 0.05 g/L EDTA, 0.2 g/L PS80, and 100 mM arginine HCl or NaCl) at pH 7 or 5.8 was compared to viscosity of formulation 1 (pH 5.0):

Sample A: formulation with 100 mM arginine HCl pH 5.8

Sample B: formulation with 100 mM NaCl pH 5.8

Sample C: formulation with 100 mM arginine HCl pH 7.0

Sample D: formulation with 100 mM NaCl pH 7.0

Sample E: control formulation 1

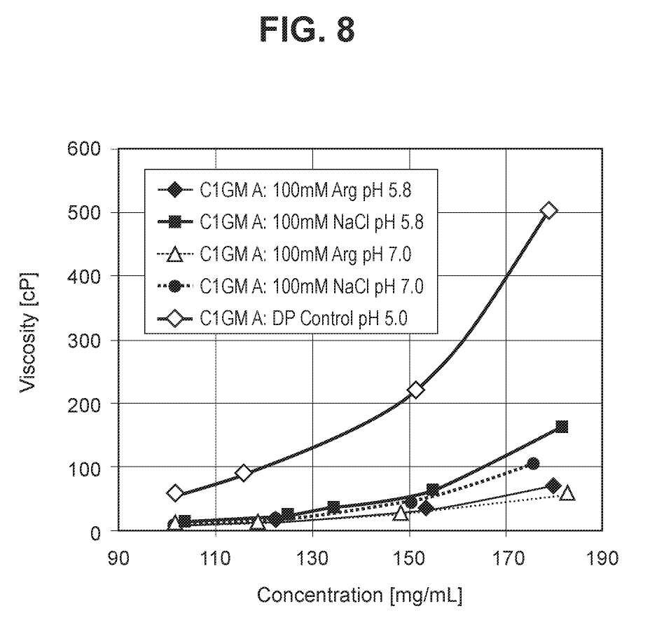

Viscosities were evaluated using an Anton-Paar rheometer in cone-plate configuration, at 25° C. The sample size was approximately 81 uL. The samples were measured with a constant shear rate (898 s-1). Results are summarized in FIG. 8.

Formulation C at pH 7.0 with 100 mM arginine HCl showed the lowest viscosity, followed by formulation A at pH 5.8 with 100 mM arginine HCl with the next lowest viscosity (FIG. 8). All formulations containing 100 mM excipient (either arginine HCl or NaCl) showed much lower viscosities than formulation 1.

Table 4 summarizes the pH of the various samples A-E.

TABLE 4

| | pH | | | |
|---|---|---|---|---|
| | | T = 8 | T = 8 | T = 8 |
| Sample | T = 0 | weeks/5° C. | weeks/25° C. | weeks/40° C. |
| A: with arginine | 6.05 | 6.05 | 6.03 | 6.05 |
| HCl pH 5.8 | | | | |
| B: with NaCl pH | 6.07 | 6.13 | 6.07 | 6.04 |
| 5.8 | | | | |
| C: with arginine | 6.88 | 6.95 | 6.89 | 6.90 |
| HCl pH 7.0 | | | | |
| D: with NaCl pH | 6.95 | 6.91 | 6.94 | 6.99 |
| 7.0 | | | | |
| E: Control pH 5.0 | 5.33 | 5.23 | 5.28 | 5.31 |
| (formulation1) | | | | |

Table 5 summarizes the mean protein concentration at T=0 of the various formulations A-E. Mean concentration at 8 weeks was 152-158 mg/mL for all samples.

TABLE 5

| Sample | Mean concentration (mg/mL) |
|---|---|
| A | 153.3 |
| B | 154.8 |
| C | 148.3 |
| D | 150.6 |
| E | 151.8 |

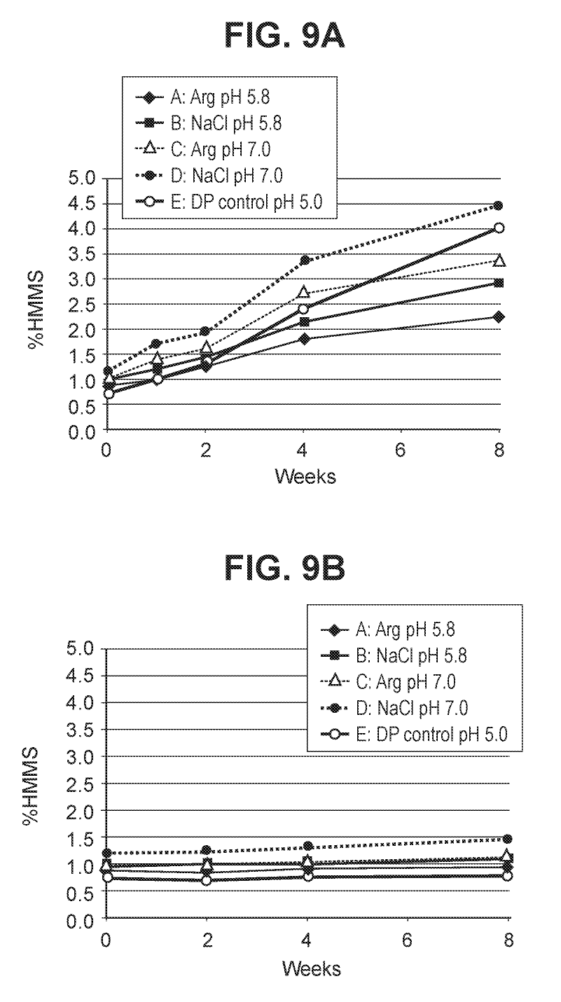

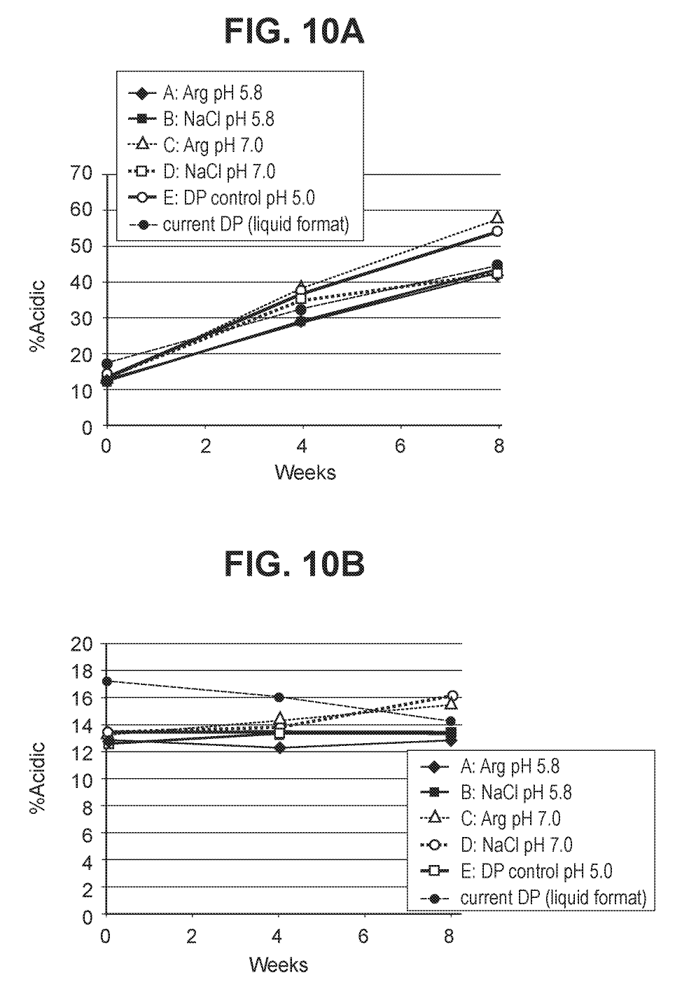

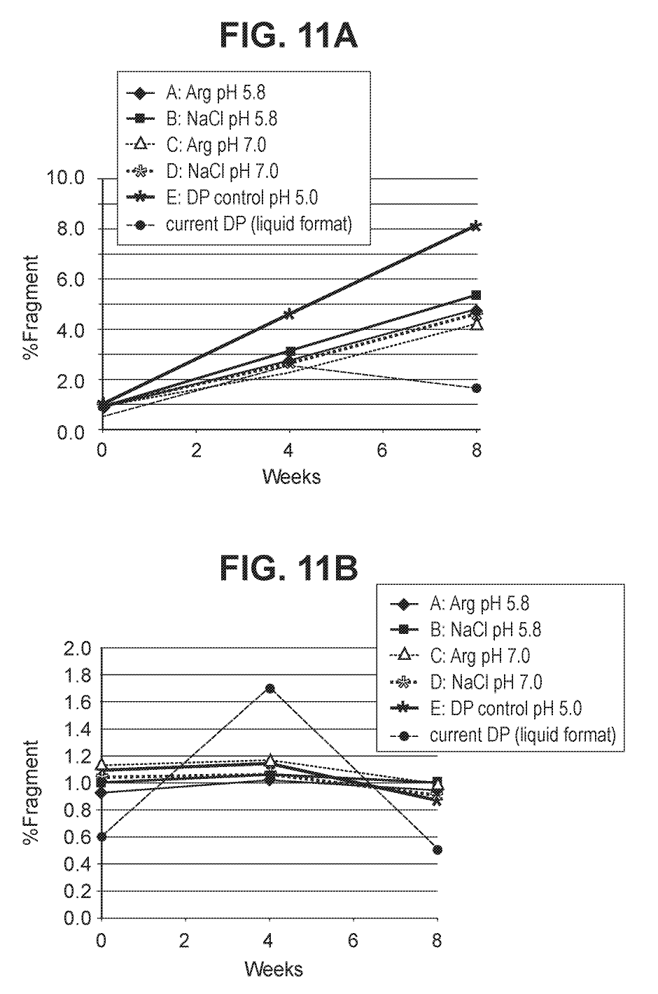

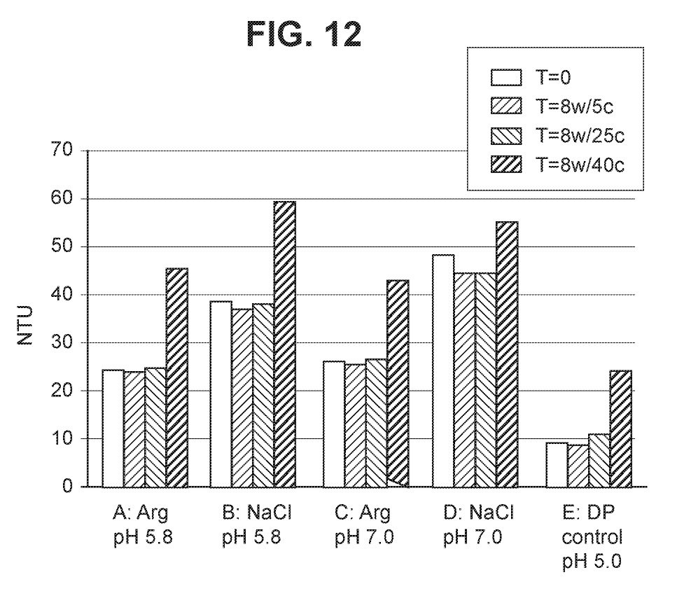

The data from the stability studies are summarized in FIGS. 9A and B (aggregation), FIGS. 10A and B (charge isoforms: acidic species), FIGS. 11A and B (fragmentation (rCGE), and FIG. 12 (turbidity (clarity)).

Osmolality was measured by freeze-point depression using samples diluted 1:1 with water. Osmolality of the undiluted samples is estimated to be approximately 400-430 mOsm/kg. The data are summarized in Table 6.

TABLE 6

| Sample | Osmolality (mOsm) |
|---|---|
| A | 188 |
| B | 202 |
| C | 197 |
| D | 190 |
| E | 177 |

These results demonstrate that the formulations showed similar stability profiles after 8 weeks. The clarity of the formulations with arginine hydrochloride was superior. The pH 7 formulation with 100 mM arginine hydrochloride showed the lowest viscosity profile of all four formulations.

### Example 8. Long-Term Stability Assessment of Anti-IL-7R Antibody Formulation

This example illustrates the stability assessment of an anti-IL-7R antibody formulation.

The formulation contains: 120 mg/mL C1GM antibody, 20 mM histidine, 100 mM Arginine HCl, 50 g/L sucrose, 0.05 g/L Disodium EDTA, 0.2 g/L PS80, pH 7.0. The formulation was prepared at 120 mg/mL C1GM antibody through dilution of 129 mg/mL drug substance with appropriate diluents to result in the target formulation. Protein stability was assessed with regard to aggregation (SEC-HPLC), fragmentation (reduced capillary electrophoresis rCGE), charge isoforms (iCE), concentration (A280) and pH. Samples were placed on long term stability for up to 3 years at 5° C. At present, 1 year of stability data is available.

The data from the stability study are summarized in Table 7.

TABLE 7

| | SEC | | | | |
|---|---|---|---|---|---|
| Assay | % | rCGE | | | |
| Timepoint | HMMS | % | iCE | Concentration | pH |
| (months) | by SEC | fragment | % acidic | mg/mL | pH |
| 0 | 0.8 | 0.7 | 15 | 120 | 7.0 |
| 1 | 0.9 | 1.1 | 14.4 | 120.1 | 7.0 |
| 2 | 1 | 1.3 | 14 | 121.1 | 7.0 |
| 3 | 1 | 1.4 | N/A | 120.7 | 7.0 |
| 4 | 1.1 | 1.2 | 15.7 | 121.9 | 7.0 |
| 6 | 1.2 | 0.7 | 15.4 | 132.9 | 7.0 |
| 12 | 1.2 | N/A | 17.5 | 118.8 | 7.0 |

These results demonstrate that this formulation (i.e., 120 mg/mL C1GM antibody, 20 mM histidine, 100 mM Arginine HCl, 50 g/L sucrose, 0.05 g/L Disodium EDTA, 0.2 g/L PS80, pH 7.0) is stable after 12 month of storage at 5° C.

1. A composition comprising;
a. an anti-IL-7R antibody, wherein the antibody concentration is between about 100 mg/ml to about 300 mg/ml,
b. arginine HCl or NaCl,
c. sucrose,
d. a buffer,
e. a chelating agent, and
f. a polysorbate,
wherein the pH of said composition is from about 6.5 to about 7.5.

2. The composition according to claim 1, wherein the concentration of sucrose is from about 1 mg/ml to about 100 mg/ml.

3. The composition according to claim 1, wherein the polysorbate is polysorbate 80 (PS80), and/or wherein the concentration of polysorbate is from about 0.01 to about 0.3 mg/ml

4. The composition according to claim 1, wherein the buffer is histidine buffer, and/or wherein the concentration of the buffer is from about 1.0 to about 30 mM.

5. The composition according to claim 1, wherein the chelating agent is disodium EDTA, and/or wherein the concentration of chelating agent ranges from about 0.01 to about 0.3 mg/mL.

6. The composition according to claim 1, wherein the antibody concentration is selected from the group consisting of about 110 mg/ml, about 115 mg/ml, about 120 mg/ml, about 125 mg/ml, about 130 mg/ml, about 135 mg/ml, and about 140 mg/ml.

7. The composition according to claim 1, comprising or consisting of:
a. about 10 mg/ml, about 105 mg/ml, about 110 mg/ml, about 115 mg/ml, about 120 mg/ml, about 125 mg/ml, about 130 mg/ml, about 135 mg/ml or about 140 mg/ml of an antibody,
b. about 20 mM histidine buffer,
c. about 100 mM arginine HCl,
d. about 50 mg/ml sucrose,
e. about 0.2 mg/ml PS80, and
f. about 0.05 mg/ml disodium EDTA,
wherein said composition is pH 7.0+/−0.5.

8. The composition according to claim 1 wherein the antibody is a human or humanized monoclonal antibody, an IgG1 or IgG2 antibody.

9. The composition according to claim 1, wherein the antibody comprises a heavy chain CDR1, CDR2, CDR3, and a light chain CDR1, CDR2, and CDR3 comprising the amino acid sequence shown in SEQ ID NO: 4, 5, 6, 7, 8, and 9, respectively.

10. The composition according to claim 1, wherein the antibody comprises an amino acid sequence that is at least 90% identical to a heavy chain variable region amino acid sequence shown in SEQ ID NO: 1, and an amino acid sequence that is at least 90% identical to a light chain variable region amino acid sequence shown in SEQ ID NO: 2

11. The composition according to claim 1, wherein the antibody comprises a variable heavy chain sequence comprising the amino acid sequence shown in SEQ ID NO: 10 and a variable light chain sequence comprising the amino acid sequence shown in SEQ ID NO: 11.

12. The composition according to claim 1, wherein the composition is lyophilized or is not lyophilized.

13. The composition according to claim 1, wherein the composition has a viscosity of about 5 to about 50 cP at 25° C.

14. Use of the composition according to claim 1, for the manufacture of a medicament for treatment of an autoimmune disorder in a mammal.

15. Use of the composition according to claim 1, for the manufacture of a medicament for treatment of an autoimmune disorder in a mammal, wherein the administration pattern of the medicament comprises administration of a dose of the medicament once every eight weeks.

16. Use according to claim 15 wherein the volume of the dose is less than or equal to about 2.5 ml, 2.0 ml, 1.5 ml, or 1.0 ml.

17. Use according to claim 15, wherein administration of the dose is either intravenous or subcutaneous.

18. Use according to claim 15, wherein the mammal is a human.
